SEQUENCE LISTING (33 sequences)

SEQ ID NO: 1 - DNA, 591 bp, Arabidopsis thaliana
  CDS (1)..(591)
atgcctattggcgttccaaaagtaccttttcgaagtcctggagaaggagatacatcttgggttgacatatacaaccgactttatcgagaaagattattttttttaggccaagaggttgataccgaaatctcgaatcaacttattagtcttatgatatatctcagtatagaaaaggataccaaagatctttatttgtttataaactctcctggtggatgggtaatatctggaatggctatttatgatactatgcaatttgtgcgacccgatgtacagacaatatgcatgggattggccgcttcaatagcatcctttatcctagtcggaggagcaattaccaaacgtatagcattccctcacgctagggtaatgatccatcaacccgctagttcgttttatgaggcacaaacgggagaatttatcttggaagcggaagaattacttaaacttcgcgaaaccatcacaagggtttatgtacaaagaacgggcaaacctatatgggttatatccgaagacatggaacgggatgtttttatgtcagcaacagaagcccaagctcatggaattgttgatcttgtagcggttcaataa

SEQ ID NO: 2 - protein, 196 aa, Arabidopsis thaliana
MPIGVPKVPFRSPGEGDTSWVDIYNRLYRERLFFLGQEVDTEISNQLISLMIYLSIEKDTKDLYLFINSPGGWVISGMAIYDTMQFVRPDVQTICMGLAASIASFILVGGAITKRIAFPHARVMIHQPASSFYEAQTGEFILEAEELLKLRETITRVYVQRTGKPIWVISEDMERDVFMSATEAQAHGIVDLVAVQ

SEQ ID NO: 3 - DNA, 1024 bp, Nicotiana tabacum
  CDS (11)..(877)
gcggccgctaatggcggtcacttttccgaccacctcttcctcgtatctacactcgagaactaaagtccctcagccttctttaagctgcgccagcaaagtttttgtcggattaagaagccaatctcctaattcttatgggattgcagcgtctaatgtaaatgttgaatttcacaatagagtgtacagaagtattgaatccggaactagagacagtaaaccaacacgtgtacgagtttccatgatgcccattgggacaccaagagtaccctacagaaatccaactgagggaacatggcagtgggttgatttgtggaatgctctttaccgtgaacgtgttattttcatcggacaacacatagatgaagaatttagcaaccagatattggcaacaatgctgtatcttgacagtattgatgattccaagaagctctacctgtatatcaatggccctggtggtgatctaactccaagcatggccatctacgacacaatgcaaagtcttaaaagtgctgttggcacccattgtgtgggctatgcctacaatcttgccggttttcttcttgctgctggagaaaagggcaatcgatttgcaatgcctctttcaaggattgcactacaatctccagctggagctgcgcgcggacaggctgatgatattcgcaatgaagcagatgaacttctcagaattagagattaccttttcaaggagttggctgagaagacaggccagcctgttgaaaaggttcacaaggatttaagtcggatgaagcgactcaatgctcaagaagctcttgaatatggtcttatagaccgtatagttaggcctccccgtattaaggcagatgctccacgaaaggataccacagcaggtcttggttagtccatacacatcgtataatttatggctgatagtggttgtacgacttgcagtgttattttgcaatttcttttgtttaatctacatattgaactcttttgatctacttattcaaaaacatgaaatcctgagcagactagcggccgc

SEQ ID NO: 4 - protein, 289 aa, Nicotiana tabacum
MAVTFPTTSSSYLHSRTKVPQPSLSCASKVFVGLRSQSPNSYGIAASNVNVEFHNRVYRSIESGTRDSKPTRVRVSMMPIGTPRVPYRNPTEGTWQWVDLWNALYRERVIFIGQHIDEEFSNQILATMLYLDSIDDSKKLYLYINGPGGDLTPSMAIYDTMQSLKSAVGTHCVGYAYNLAGFLLAAGEKGNRFAMPLSRIALQSPAGAARGQADDIRNEADELLRIRDYLFKELAEKTGQPVEKVHKDLSRMKRLNAQEALEYGLIDRIVRPPRIKADAPRKDTTAGLG

SEQ ID NO: 5 - DNA, 1124 bp, Arabidopsis thaliana
  CDS (2)..(931)
aatggagatgagtttgcgtctcgcttcatcttcaacctcaaacccaatttgtctactaaaccctggaaaaaaccttaatttcccaatccgaaaccatagaatccctaaaacttcgaaacccttttgcgttaggtcttcaatgagcttgtctaaaccacccagacaaaccttatctagtaactgggatgtatctagcttctccattgattccgttgctcaatctccttcaagactcccaagtttcgaagaactcgataccaccaacatgttgctccgtcaaagaatcgtctttttgggttctcaggttgatgatatgacggcggatttggttataagtcagctattgttactagatgctgaggactcagaaagagacattacgctttttatcaattcacccggtggatctattactgctgggatgggaatatatgatgcaatgaaacaatgtaaggcggatgtatctactgtttgcttagggttagctgcatctatgggtgcgtttcttcttgcttctggttcaaaagggaaacggtattgtatgcctaactctaaagttatgatccatcagccacttggtactgctggaggcaaagcaacggaaatgagcatacgtataagagaaatgatgtaccacaagattaaacttaacaaaatcttctctagaatcactgggaagcctgaatcagagatcgaaagtgacacagaccgtgataacttcttgaatccatgggaggcgaaagaatatggtttgatcgacgctgtaatcgatgatgggaaaccgggactaatcgctccaattggagatggtactcctcctcctaaaaccaaagtctgggatctttggaaagtcgaaggaaccaagaaagacaacactaacttgccatctgagcgctccatgacacagaatggttatgccgccattgaatagaactgttgttgcagcgtttacgccttttatatgttattctggtggtacctgtaaccatataacgttgcatttcctgtgtttgtaccatttctctgatagattttggaataatttgaaggcaaagatagattattgtgtagagagctacaaatttaatgataaattgatcatcagcactggaaagctaaaaaaa

SEQ ID NO: 6 - protein, 309 aa, Arabidopsis thaliana
MEMSLRLASSSTSNPICLLNPGKNLNFPIRNHRIPKTSKPFCVRSSMSLSKPPRQTLSSNWDVSSFSIDSVAQSPSRLPSFEELDTTNMLLRQRIVFLGSQVDDMTADLVISQLLLLDAEDSERDITLFINSPGGSITAGMGIYDAMKQCKADVSTVCLGLAASMGAFLLASGSKGKRYCMPNSKVMIHQPLGTAGGKATEMSIRIREMMYHKIKLNKIFSRITGKPESEIESDTDRDNFLNPWEAKEYGLIDAVIDDGKPGLIAPIGDGTPPPKTKVWDLWKVEGTKKDNTNLPSERSMTQNGYAAIE

SEQ ID NO: 7 - DNA, 1183 bp, Arabidopsis thaliana
  CDS (3)..(902)
ctttcttcttcttcgcttcagccatgggaaccctatctctctcatcttctctcaaaccttcactcgtttcatcaagactcaattcatcttcctccgcatcttcttcttcgtttcctaaaccaaacaatctctacctcaaacccaccaaactcatttcaccacctctcagaacaacttcaccatcgccattgagattcgccaatgcttcaatcgagatgtcgcagacacaggaatcagctattcgcggagctgaatctgacgtcatgggtcttctccttagggaacgaatcgtctttctcggtagtagtatcgacgatttcgtcgctgatgctattatgagtcagttgcttctcttagatgctaaagatccaaagaaagatatcaaactctttatcaattctcctggtggttctctcagtgcaaccatggctatatacgatgtggttcagcttgtgagagctgatgtttcgacgattgctcttggcattgctgcatcaacagcttcgattattcttggtgcgggaactaaaggcaagcgctttgctatgcccaacacgaggataatgattcatcaacctcttggaggtgcaagcggtcaagctatagatgttgagattcaagctaaggaagttatgcataacaagaacaatgtcaccagcattatcgcgggatgtactagtcgatcgtttgagcaggttctgaaagatattgatagggaccggtacatgtctccaattgaagcagttgagtatggtttaattgatggagttattgatggagacagcattattcctcttgaacctgttcctgatagagtgaaaccgagagtaaactacgaggagattagcaaggatccgatgaaattcttgactcccgagatacctgatgatgagatctactaaagccaagctcgtctagaagcagggatcttcaaatgtgactaagactagcagtttcgaggaaaagctcaatttcttctgcggttactggtattggctttgcgaaaccgaagctggtagtacttggcttttgtatctcatatttcagttgttcagaaaataattgttctttaaatcactctgttttgaggaaaatgacttaaagaagctgtagttatctcgtttatgacaatcccttcaagtgtttaatggattcaagaagtatcagtcagtatttttgtggt

SEQ ID NO: 8 - protein, 299 aa, Arabidopsis thaliana
FFFFASAMGTLSLSSSLKPSLVSSRLNSSSSASSSSFPKPNNLYLKPTKLISPPLRTTSPSPLRFANASIEMSQTQESAIRGAESDVMGLLLRERIVFLGSSIDDFVADAIMSQLLLLDAKDPKKDIKLFINSPGGSLSATMAIYDVVQLVRADVSTIALGIAASTASIILGAGTKGKRFAMPNTRIMIHQPLGGASGQAIDVEIQAKEVMHNKNNVTSIIAGCTSRSFEQVLKDIDRDRYMSPIEAVEYGLIDGVIDGDSIIPLEPVPDRVKPRVNYEEISKDPMKFLTPEIPDDEIY

SEQ ID NO: 9 - DNA, 1056 bp, Arabidopsis thaliana
  CDS (61)..(876)
gagtaatttagcatctatccacgcctgaacccgaaaaactctgaaagctgagctctggtaatggcgggtttagcaatttcacctcctctcggtctttccttctcttctcgaactcgaaaccctaaacccacttcctttctatctcacaatcaaaggaatcctataagacgtatagtttctgctctacagagtccatatggagattctctgaaagctggactttctagtaatgtttctggatccccaataaagattgacaacaaggctccaagatttggagtgatagaggcgaaaaagggaaaccccccagtaatgccttcagtgatgacccctggaggacctttagacctctcttctgtgttattccgtaaccgcataatcttcatcgggcaaccaattaacgcacaggttgctcagcgagtcatatctcagcttgtaacccttgcatctattgatgataaatccgacatcctgatgtacttgaattgtcccggtggcagtacttactccgtcctaacaatttatgactgtatgtcttggataaagcctaaagttggaacagtggcgtttggagtagctgcaagccaaggagcattttttcttgctggaggtgaaaaaggaatgcgttatgcaatgccaaatactcgtgtcatgatacatcaaccacaaactggatgcggaggacatgtagaggacgtgaggagacaggtcaatgaagccatcgaagcccgacaaaaaattgacaggatgtatgcagctttcactggacaacctctggagaaagtgcagcaatacactgaaagagatcgtttcttatcagcatctgaggcgtttgagttcgggctcattgatggtctattggaaacagaatactgaagcagcatacaggacaatgcacaacaacagctcattgcaatgttcaaagcttccattttcatttgaatatgaacggttgtaactgatatttgtgcataaatcagtttggttttcttggttttattgtctactaaacagaatgagaaaactaaactgtttatttttttactgaaaaatctg

SEQ ID NO: 10 - protein, 271 aa, Arabidopsis thaliana
MAGLAISPPLGLSFSSRTRNPKPTSFLSHNQRNPIRRIVSALQSPYGDSLKAGLSSNVSGSPIKIDNKAPRFGVIEAKKGNPPVMPSVMTPGGPLDLSSVLFRNRIIFIGQPINAQVAQRVISQLVTLASIDDKSDILMYLNCPGGSTYSVLTIYDCMSWIKPKVGTVAFGVAASQGAFFLAGGEKGMRYAMPNTRVMIHQPQTGCGGHVEDVRRQVNEAIEARQKIDRMYAAFTGQPLEKVQQYTERDRFLSASEAFEFGLIDGLLETEY

SEQ ID NO: 11 - DNA, 1448 bp, Nicotiana tabacum
  CDS (2)..(1162)
gcggccgctggcttcttctttgcttctctctccgctttctagctcgacggttactgaaaatcgcgagctgggttctggtaaatcaactttcatatccagtcccaatttctcctttgcaacttctgttcacagttgcaggccaaacggcgttcgaggttattgttacaggtctccggtagctaagtctttggaccatataccccaaaaattcagactggaaaatctcaaagatggactactggacaactataaaagtgcccctcagtatctttacggccttagtccttcacagatggatatgttcatgacagaagataacccagtacggcgacagtcagaaagtgccactgaggatagtatatcttcagccaataactatctgagcaatggtggaatgtggagtatgtccggcatgaacgatcggggcccctcgaaatacagtatgagtgtcagcatgtaccgtggaggaacaagaggatctggaagacctcgaactgcgcctcctgatttgccatctttgcttttggatgctcgaattgtctatctgggcatgcctattgtaccagctgttacagagcttcttgttgctcagtttatgtggttggattatgacaatccatcaaagcctatatacctatatataaactcatcaggcacacagaatgagaagatggagactgttgggtctgaaacagaggcatatgccatcgctgacacaatggcatactgcaaatcagatatctatacagtgaactgtggcatggcatatggtcaagcagcaatgcttctgtcactgggaaagaaggggttccgtgctatgcagccaaattcatctacaaaattgtatttacctaaagtcagcaaatccagtggagcagtgatagatatgtggatcagggccaaagaactagatgcaaacactgagtattaccttgaactattagcgaaaggagttggaaaaccaaaggaagaaatcgagaaagatattcaacgccctaaatatctgcgggcacaagaagccattgactatggcattgcggacaagataatcgattcaagagacaatgcatttgagaaaaggaactatggtgagatactcgcccaatctagagctatgaggaaagccggaccaggtgctcaggctgctccatctggctccaggtgactggaagagcggtaatggtcccaagctttcaggaacaactgttgttcccttatagtttcgaggaacaaagttgctggttacttggtctgtgccggtataatgtaactgggacaaagaacatattgtagaaaccttgtttgagctgtgaagtataggggttttacaactattatgcacaggtctgcaaagagtacccataatgtcaattggttgtaccagtatcaaacaatcagatagtgccagtgtatggtataaatgaatatagatctctctgagcggccgc

SEQ ID NO: 12 - protein, 387 aa, Nicotiana tabacum
RPLASSLLLSPLSSSTVTENRELGSGKSTFISSPNFSFATSVHSCRPNGVRGYCYRSPVAKSLDHIPQKFRLENLKDGLLDNYKSAPQYLYGLSPSQMDMFMTEDNPVRRQSESATEDSISSANNYLSNGGMWSMSGMNDRGPSKYSMSVSMYRGGTRGSGRPRTAPPDLPSLLLDARIVYLGMPIVPAVTELLVAQFMWLDYDNPSKPIYLYINSSGTQNEKMETVGSETEAYAIADTMAYCKSDIYTVNCGMAYGQAAMLLSLGKKGFRAMQPNSSTKLYLPKVSKSSGAVIDMWIRAKELDANTEYYLELLAKGVGKPKEEIEKDIQRPKYLRAQEAIDYGIADKIIDSRDNAFEKRNYGEILAQSRAMRKAGPGAQAAPSGSR

SEQ ID NO: 13 - DNA, 1246 bp, Arabidopsis thaliana
  CDS (38)..(1030)
attttcgcgagcttccgtgtccaagagctcctcgaccatggcgtcttgtttacaagcatccatgaattctctgcttccacgctcttcttctttttctcctcatcctcctctatcttcgaattcatccgggagaagaaacttgaagacttttcgttacgcctttcgcgccaaagcctctgccaaaatccctatgcctccgataaatccaaaggatcctttcctctccacgctcgcttctattgccgcgaattctccggaaaagcttctcaatcggccggttaacgctgatgtgccgccatatcttgacatctttgactcccctcagctcatgtcttctcctgcacaggttgaaagatcagtggcttataacgagcaccgaccgagaactcctccaccagacttgccatctatgcttcttgacgggagaattgtttacattggaatgcctcttgtgccggcagtgactgagctagttgtcgctgagctaatgtatcttcagtggctggatcccaaggaacccatttacatttacatcaactccacagggaccactcgtgatgatggagagacggttggaatggaatcagaagggtttgcgatctatgactctttgatgcaacttaaaaacgaggtacatacagtatgtgtgggagcagccataggtcaggcctgtctattactttctgcgggaaccaagggtaaacggtttatgatgccacatgccaaagcgatgattcagcaaccacgtgtaccttcttctgggttgatgcctgccagtgatgtcctgattcgggccaaagaggtcattacaaatagggatatacttgtggaactactatcaaagcatactgggaattccgtggagactgtagctaacgtaatgagaaggccatattacatggatgcaccaaaagctaaagaatttggagtcattgacaggattctttggcgcggtcaagaaaagattattgcggacgtggttccttcagaggaattcgacaagaatgcagggattaaaagcgtagtatgagtctagtcttaagttttcttggcctaaatcatactgcgtcatggagaagaacaaatagactgaccaaaatcacattggccgcagactgccttgtttcaaatcacttggtaaatgtgaacatgcgattaggagaatcatacttaaaggatcttgaaatattatgataaaattgtaatgtgtttgttcgttagcaatagtaaatacaatcttcaactc

SEQ ID NO: 14 - protein, 330 aa, Arabidopsis thaliana
MASCLQASMNSLLPRSSSFSPHPPLSSNSSGRRNLKTFRYAFRAKASAKIPMPPINPKDPFLSTLASIAANSPEKLLNRPVNADVPPYLDIFDSPQLMSSPAQVERSVAYNEHRPRTPPPDLPSMLLDGRIVYIGMPLVPAVTELVVAELMYLQWLDPKEPIYIYINSTGTTRDDGETVGMESEGFAIYDSLMQLKNEVHTVCVGAAIGQACLLLSAGTKGKRFMMPHAKAMIQQPRVPSSGLMPASDVLIRAKEVITNRDILVELLSKHTGNSVETVANVMRRPYYMDAPKAKEFGVIDRILWRGQEKIIADVVPSEEFDKNAGIKSVV

SEQ ID NO: 15 - DNA, 1236 bp, Arabidopsis thaliana
  CDS (66)..(983)
agatcgttatcgtttcggggtcacagggactttcactctttctctctctctgcaacaaagaagaaatggaggtagcagcagcgactgcgacgagcttcacaacgcttcgagctcgtacgtcagcgattatcccgtcttctacacgtaatctgagatctaaaccgagattttcttcatcttcatctctcagagcttctctttcgaatggctttctttcgccgtataccggaggaagcatctctagtgacttatgcggcgctaagcttcgtgcggaatcgcttaatccgttaaatttttccagttccaagcctaaacgcggagttgtcactatggttatacctttctcaaagggaagtgcacacgaacaacctcctcctgatttggcatcatatttgttcaagaaccgaattgtatatttgggaatgtctctcgtaccttcagttactgagttgatacttgcggagtttctttaccttcagtatgaagacgaggaaaagcctatttacctttacataaactcgactgggacaaccaagaatggtgaaaagttgggctatgatactgaggcttttgcaatctatgatgtcatggggtatgtcaaaccaccaatctttactctttgcgtcgggaatgcgtggggtgaagctgctttgcttctgactgctggtgcaaaaggaaatcgatctgcgttgccctcatcaactattatgataaagcagcccattgctcgatttcaaggccaagcaactgatgttgaaattgcaaggaaagaaatcaagcacataaagacagaaatggtcaagctgtattcaaagcatattggtaaatccccggagcagattgaagctgacatgaaacgcccgaaatattttagtcccactgaggctgttgaatatgggatcattgataaggtggtttacaatgaaaggggcagccaagacagaggagttgtgtctgaccttaaaaaggcacaactcatttgaatgtcagaactgtcttccgaaatcccatgattaacaggttggagatcttaccgctgatcaaatggggaatcagtgaaccattcaccggcacagaactgaggtaaagtctggaaaacatgttaaaaaaggttactagtaatgctgcaattgtagggttatttgaacagaaacaaacccatatgtgtaggcttgtgaatgcctagaaacaggattggtgtatcttcaatatatgtttctaagatgaatcaatttc

SEQ ID NO: 16 - protein, 305 aa, Arabidopsis thaliana
MEVAAATATSFTTLRARTSAIIPSSTRNLRSKPRFSSSSSLRASLSNGFLSPYTGGSISSDLCGAKLRAESLNPLNFSSSKPKRGVVTMVIPFSKGSAHEQPPPDLASYLFKNRIVYLGMSLVPSVTELILAEFLYLQYEDEEKPIYLYINSTGTTKNGEKLGYDTEAFAIYDVMGYVKPPIFTLCVGNAWGEAALLLTAGAKGNRSALPSSTIMIKQPIARFQGQATDVEIARKEIKHIKTEMVKLYSKHIGKSPEQIEADMKRPKYFSPTEAVEYGIIDKVVYNERGSQDRGVVSDLKKAQLI

SEQ ID NO: 17 - DNA, 906 bp, Nicotiana tabacum
  CDS (45)..(755)
gcggccgctccaagattcatccccaactctcaacacattcaactatgcgcacccaaattgttcacaaactctttaaccgaagaatcaacggaacccctttgaatagtagtaagagattttatggggtaataccaatggtaatagagcactcttcaagaggagaaagggcttatgacatattctcaaggctattaaaggaacgaattatttgcattaacggccccattgatgattccacttctcatgttgttgttgctcagcttctttttcttgaatctgagaacccttctaagcctattcacaagtacctcaactctccaggtggcgctgttacagctggtcttgcaatctatgataccacgcagtatatccgatctccaattcatactatatgcctaggtcaagcagcttcaatgggatcccttctcttagctgcaggtgcaaagggtgagagacgatctctccctaatgcttcagttatgattcaccagcctttcggtgggtatagcgggcaggctaaagatttgacgatccacacaaaacagatagttcgggtatgggatactttgaatgacctatatgcaaagcatacaggacaacctatagaaataattcaaaagaatatggatagggattatttcatgacacctgaagaggcgaaggagtttggaataatcgatgaagttatagatgaacgaccaatggctttagtaactgatgctgttgcaaatgaagccaaagaaaaaggttcaagctagaaaaattgctgtaatactgatctcattgcagtctttgttagcatttaccatcgctaactagttctccattttacttactggtgtatttactttctagtattttatttgatgaggcgatacctcattactttgttttctcagcggccgc

SEQ ID NO: 18 - protein, 237 aa, Nicotiana tabacum
MRTQIVHKLFNRRINGTPLNSSKRFYGVIPMVIEHSSRGERAYDIFSRLLKERIICINGPIDDSTSHVVVAQLLFLESENPSKPIHKYLNSPGGAVTAGLAIYDTTQYIRSPIHTICLGQAASMGSLLLAAGAKGERRSLPNASVMIHQPFGGYSGQAKDLTIHTKQIVRVWDTLNDLYAKHTGQPIEIIQKNMDRDYFMTPEEAKEFGIIDEVIDERPMALVTDAVANEAKEKGSS

SEQ ID NO: 19 - DNA, 447 bp, Nicotiana tabacum
gcggccgcttgcggacaagataatcgattcaagagacaatgtatttgagaaaaggaactatgatgagatactcgcccaatctagagctatgaggaaagccggaccaggtgctcaggctgctccatctggcttcaggtgactggaagagcggtaatggtcccaaactttcaggaacaactgttgttcccttatagtttcgaggaacaaagttgctggttacttggtctgtgccggtataatgtaactgggacaaagaacatattgtagaaaccttgtttgagctgtgaagtataggggttttacaactattatgcacaggtctgcaaagagtacccataatgtcaattggttgtaccagtatcaaacaatcagatagtgccagtgtatggtataaatgaatatagatctctctgatgtcatttttcttttatcatgttcagcggccgc

SEQ ID NO: 20 - DNA, 996 bp, Nicotiana tabacum
gcggccgcttgcggacaagataatcgattcaagagacaatgtatttgagaaaaggaactatgatgagatactcgcccaatctagagctatgaggaaagccggaccaggtgctcaggctgctccatctggcttcaggtgactggaagagcggtaatggtcccaaactttcaggaacaactgttgttcccttatagtttcgaggaacaaagttgctggttacttggtctgtgccggtataatgtaactgggacaaagaacatattgtagaaaccttgtttgagctgtgaagtataggggttttacaactattatgcacaggtctgcaaagagtacccataatgtcaattggttgtaccaggcggccgctggcttcttctttgcttctctctccgctttctagctcgacggttactgaaaatcgcgagctgggttctggtaaatcaactttcatatccagtcccaatttctcctttgcaacttctgttcacagttgcaggccaaacggcgttcgaggttattgttacaggtctccggtagctaagtctttggaccatataccccaaaaattcagactggaaaatctcaaagatggactactggacaactataaaagtgcccctcagtatctttacggccttagtccttcacagatggatatgttcatgacagaagataacccagtacggcgacagtcagaaagtgccactgaggatagtatatctgcggccgctggcagatgctccacgaarggataccacagcaggtcttggttagtccatacacatcgtataatttatggctgatagtggttgtacgacttgcagtgttattttgcaatttcttttgtttaatctacatattgaactcttttgatctacttattcaaaaacatgaaatcctgagcagactagatgcatttgtttaatatcatgaatgcaaggaatccacctacagctgatatgtatacaaagatacctttttttcaagagcggccgc

SEQ ID NO: 21 - DNA, 602 bp, Nicotiana tabacum
  CDS (2)..(193)
gcggccgctggaagatgtgcggcgccaagtgaacgaagcggttcaacctcgtcataaaatcgacaagatgtatgtcgcctttactgaccaaccaattgagaaggtgcaacagtacactgaaagggatcgttttttgtctgtctcagaggccatggagtttggtctcatagatggggtgctagaaacagaatactagttgcaaatgaatctttagtagtacatggtagctagccttccaatgacgaaaaagctggtgttgctcattaaccacttcgaagtacaagaagctggctcttgcaaatttgtatcgtagaaatatctcaactcttcaatccaggaatgtccaaaagcctaattctgaagacggttatagaaagcgctcttgttttactatttttgtctctcctgcagatacactcagcacttttgtgggtattaatcagggtcttaattcatcacttaatcacaatccagttggaagcgaagtgatcaaacacaaagcagattcaggaagatgtgtatttttcccaaatatatattactccaattgctatcatcccttcgctgtcgttatgaaaggatatttattttatagcggccgc

SEQ ID NO: 22 - protein, 64 aa, Nicotiana tabacum
RPLEDVRRQVNEAVQPRHKIDKMYVAFTDQPIEKVQQYTERDRFLSVSEAMEFGLIDGVLETEY

SEQ ID NO: 23 - DNA, 16 bp, artificial sequence
  primer
agaattcgcggccgct

SEQ ID NO: 24 - DNA, 32 bp, artificial sequence
  primer
ctcatgcggccgcgcgcaacgcaattaatgtg

SEQ ID NO: 25 - DNA, 32 bp, artificial sequence
  primer
tcatgcggccgcgagatccagttcgatgtaac

SEQ ID NO: 26 - DNA, 21 bp, artificial sequence
  primer
gtggattgatgtgatatctcc

SEQ ID NO: 27 - DNA, 21 bp, artificial sequence
  primer
gtaaggatctgagctacacat

SEQ ID NO: 28 - DNA, 24 bp, artificial sequence
  primer
tataccatggatttgccatctttg

SEQ ID NO: 29 - DNA, 21 bp, artificial sequence
  primer
atagatctcacctggagccag

SEQ ID NO: 30 - DNA, 19 bp, artificial sequence
  primer
gagcccatggcaagaggag

SEQ ID NO: 31 - DNA, 22 bp, artificial sequence
  primer
atagatctttctagcttgaacc

SEQ ID NO: 32 - DNA, 21 bp, artificial sequence
  primer
tcagccatggcccctggagga

SEQ ID NO: 33 - DNA, 24 bp, artificial sequence
  primer
taagatcttcagtattctgtttcc

(19) United States
(12) Patent Application Publication
(10) Pub. No.: US 2007/0113300 A1
(43) Pub. Date: May 17, 2007

(54) Clp-protease as target for herbicides

(75) Inventors: Thomas Ehrhardt, Speyer (DE); Andreas Reindl, Mannheim (DE); Annette Freund, Limburgerhof (DE); Ralf-Michael Schmidt, Kirrweiler (DE); Uwe Sonnewald, Quedlinburg (DE); Marc Stitt Nigel, Potsdam (DE); Wolfgang Lein, Potsdam (DE); Frederik Bornke, Quedlinburg (DE); Kirsten Deist, Westdorf (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/581,293

(22) PCT Filed: Nov. 30, 2004
(86) PCT No.: PCT/EP04/13555

§ 371 Date: Jun. 1, 2006

(30) Foreign Application Priority Data
Dec. 2, 2003 (EP) 03027637.2

(51) Int. Cl. A01H 1/00; C12Q 1/37; C12P 21/06; A01N 25/00; C12N 9/48; C12N 5/04; C12N 15/82; C07H 21/04

(52) U.S. Cl. 800/278; 435/23; 435/69.1; 435/212; 435/419; 435/468; 504/116.1; 536/23.2

(57) ABSTRACT

The present invention relates to Clp-protease, which, when absent, brings about reduced growth and chlorotic leaves as target for herbicides. For this purpose, novel nucleic acid sequences encompassing SEQ ID NO:3, SEQ ID NO:11 and SEQ ID NO: 17 and functional equivalents of SEQ ID NO:3, SEQ ID NO:11 and SEQ ID NO: 17 are provided. Moreover, the present invention relates to the use of Clp-protease in a method for identifying compounds with herbicidal or growth-regulatory activity, and to the use of the compounds identified by this method as herbicides or growth regulators.

The present invention relates to Clp-protease, which, when absent, brings about reduced growth and chlorotic leaves as target for herbicides. For this purpose, novel nucleic acid sequences encompassing SEQ ID NO:3, SEQ ID NO:11 and SEQ ID NO: 17 and functional equivalents of SEQ ID NO:3, SEQ ID NO:11 and SEQ ID NO: 17 are provided. Moreover, the present invention relates to the use of Clp-protease in a method for identifying compounds with herbicidal or growth-regulatory activity, and to the use of the compounds identified by this method as herbicides or growth regulators.

The basic principle of identifying herbicides via the inhibition of a defined target is known (for example U.S. Pat. No. 5,187,071, WO 98/33925, WO 00/77185). In general, there is a great demand for the detection of enzymes which might constitute novel targets for herbicides. The reasons are resistance problems which occur with herbicidal active ingredients which act on known targets, and the ongoing endeavor to identify novel herbicidal active ingredients which are distinguished by as wide as possible a spectrum of action, ecological and toxicological acceptability and/or low application rates.

In practice, the detection of novel targets entails great difficulties since the inhibition of an enzyme which forms part of a metabolic pathway frequently has no further effect on the growth of the plant. This may be attributed to the fact that the plant switches to alternative metabolic pathways whose existence is not known or that the inhibited enzyme is not limiting for the metabolic pathway. Furthermore, plant genomes are distinguished by a high degree of functional redundancy. Functionally equivalent enzymes are found more frequently in gene families in the Arabidopsis thaliana genome than in insects or mammals (Nature, 2000, 408(6814):796-815). This hypothesis is confirmed experimentally by the fact that comprehensive gene knock-out programs by T-DNA or transposon insertion into Arabidopsis yielded fewer manifested phenotypes to date than expected (Curr. Op. Plant Biol. 4, 2001, pp. 111-117).

It is an object of the present invention to identify novel targets which are essential for the growth of plants or whose inhibition leads to reduced plant growth, and to provide methods which are suitable for identifying herbicidally active and/or growth-regulatory compounds.

We have found that this object is achieved by the use of nuclear encoded Clp-protease in a method for identifying herbicides.

Further terms used in the description are now defined at this point.

“Affinity tag”: this refers to a peptide or polypeptide whose coding nucleic acid sequence can be fused to the nucleic acid sequence according to the invention either directly or by means of a linker, using customary cloning techniques. The affinity tag serves for the isolation, concentration and/or selective purification of the recombinant target protein by means of affinity chromatography from total cell extracts. The abovementioned linker can advantageously contain a protease cleavage site (for example for thrombin or factor Xa), whereby the affinity tag can be cleaved from the target protein when required. Examples of common affinity tags are the “His tag”, for example from Qiagen, Hilden, “Strep tag”, the “Myc tag” (Invitrogen, Carlsberg), the tag from New England Biolabs which consists of a chitin-binding domain and an inteine, the maltose-binding protein (pMal) from New England Biolabs, and what is known as the CBD tag from Novagen. In this context, the affinity tag can be attached to the 5′ or the 3′ end of the coding nucleic acid sequence with the sequence encoding the target protein.

“Activity of nuclear encoded Clp-protease”: the term activity describes the ability of an enzyme to convert a substrate into a product. The enzymatic activity can be determined in what is known as an activity assay via the increase in the product, the decrease in the substrate (or starting material) or the decrease in a specific cofactor, or via a combination of at least two of the abovementioned parameters, as a function of a defined period of time. “Activity of nuclear encoded Clp-protease” describes here the ability of an enzyme to catalyze the hydrolysis of peptides of maximal five amino acids in vitro.

“Expression cassette”: an expression cassette contains a nucleic acid sequence according to the invention linked operably to at least one genetic control element, such as a promoter, and, advantageously, a further control element, such as a terminator. The nucleic acid sequence of the expression cassette can be for example a genomic or complementary DNA sequence or an RNA sequence, and their semisynthetic or fully synthetic analogs. These sequences can exist in linear or circular form, extrachromosomally or integrated into the genome. The nucleic acid sequences in question can be synthesized or obtained naturally or contain a mixture of synthetic and natural DNA components, or else consist of various heterologous gene segments of various organisms.

Artificial nucleic acid sequences are also suitable in this context as long as they make possible the expression, in a cell or an organism, of a polypeptide with the enzymatic activity of a nuclear encoded Clp Protease, preferably with the biological activity of a a nuclear encoded Clp Protease, which polypeptide is encoded by a nucleic acid sequence according to the invention. For example, synthetic nucleotide sequences can be generated which have been optimized with regard to the codon usage of the organisms to be transformed.

All of the abovementioned nucleotide sequences can be generated from the nucleotide units by chemical synthesis in the manner known per se, for example by fragment condensation of individual overlapping complementary nucleotide units of the double helix. Oligonucleotides can be synthesized chemically for example in the manner known per se using the phosphoamidite method (Voet, Voet, 2nd Edition, Wiley Press New York, pp. 896-897). When preparing an expression cassette, various DNA fragments can be manipulated in such a way that a nucleotide sequence with the correct direction of reading and the correct reading frame is obtained. The nucleic acid fragments are linked with each other via general cloning techniques as are described, for example, in T. Maniatis, E. F. Fritsch and J. Sambrook, “Molecular Cloning: A Laboratory Manual”, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., “Current Protocols in Molecular Biology”, Greene Publishing Assoc. and Wiley-Interscience (1994).

“Operable linkage” or “functional linkage”: an operable, or functional, linkage is understood as meaning the sequential arrangement of regulatory sequences or genetic control elements in such a way that each of the regulatory sequences, or each of the genetic control elements, can fulfill its intended function when the coding sequence is expressed.

“Functional equivalents” describe, in the present context, nucleic acid sequences which hybridize under standard conditions with the nucleic acid sequence SEQ ID NO:1, SEQ ID NO:3, SEQ ID NO:5, SEQ ID NO:7, SEQ ID NO:9, SEQ ID NO:11, SEQ ID NO:3, SEQ ID NO:13, SEQ ID NO:15 or SEQ ID NO:17 or parts of the aforementioned nucleic acid sequences and which are capable of bringing about the expression, in a cell or an organism, of a polypeptide with the activity of Clp protease.

To carry out the hybridization, it is advantageous to use short oligonucleotides with a length of approximately 10-50 bp, preferably 15-40 bp, for example of the conserved or other regions, which can be determined in the manner with which the skilled worker is familiar by comparisons with other related genes. However, longer fragments of the nucleic acids according to the invention with a length of 100-500 bp, or the complete sequences, may also be used for hybridization. Depending on the nucleic acid/oligonucleotide used, the length of the fragment or the complete sequence, or depending on which type of nucleic acid, i.e. DNA or RNA, is being used for the hybridization, these standard conditions vary. Thus, for example, the melting temperatures for DNA:DNA hybrids are approximately 10° C. lower than those of DNA: RNA hybrids of the same length.

Standard hybridization conditions are to be understood as meaning, depending on the nucleic acid, for example temperatures of between 42 and 58oC in an aqueous buffer solution with a concentration of between 0.1 and 5×SSC (1×SSC=0.15 M NaCl, 15 mM sodium citrate, pH 7.2) or additionally in the presence of 50% formamide, such as, for example, 42° C. in 5×SSC, 50% formamide. The hybridization conditions for DNA:DNA hybrids are advantageously 0.1×SSC and temperatures of between approximately 20° C. and 65° C., preferably between approximately 30° C. and 45° C. In the case of DNA:RNA hybrids, the hybridization conditions are advantageously 0.1×SSC and temperatures of between approximately 30° C. and 65° C., preferably between approximately 45° C. and 55° C. These hybridization temperatures which have been stated are melting temperature values which have been calculated by way of example for a nucleic acid with a length of approx. 100 nucleotides and a G+C content of 50% in the absence of formamide. The experimental conditions for DNA hybridization are described in relevant textbooks of genetics such as, for example, in Sambrook et al., “Molecular Cloning”, Cold Spring Harbor Laboratory, 1989, and can be calculated using formulae with which the skilled worker is familiar, for example as a function of the length of nucleic acids, the type of the hybrids or the G+C content. The skilled worker will find further information on hybridization in the following textbooks: Ausubel et al. (eds), 1985, “Current Protocols in Molecular Biology”, John Wiley & Sons, New York; Hames and Higgins (eds.), 1985, “Nucleic Acids Hybridization: A Practical Approach”, IRL Press at Oxford University Press, Oxford; Brown (ed.), 1991, Essential Molecular Biology: A Practical Approach, IRL Press at Oxford University Press, Oxford.

A functional equivalent of SEQ ID NO:1, SEQ ID NO:3, SEQ ID NO:5, SEQ ID NO:7, SEQ ID NO:9, SEQ ID NO:11, SEQ ID NO:3, SEQ ID NO:13, SEQ ID NO:15 or SEQ ID NO:17 can be furthermore defined by the degree of homology or identity with SEQ ID NO:1, SEQ ID NO:3, SEQ ID NO:5, SEQ ID NO:7, SEQ ID NO:9, SEQ ID NO:11, SEQ ID NO:3, SEQ ID NO:13, SEQ ID NO:15 or SEQ ID NO:17, respectively, and can furthermore comprise also natural or artificial mutations of the aforementioned nucleic acid sequences which encode a polypeptide with the activity of a nuclear encoded Clp-protease.

The present invention also encompasses, for example, those nucleotide sequences which are obtained by modification of the SEQ ID NO:1, SEQ ID NO:3, SEQ ID NO:5, SEQ ID NO:7, SEQ ID NO:9, SEQ ID NO:11, SEQ ID NO:3, SEQ ID NO:13, SEQ ID NO:15 or SEQ ID NO:17.

For example, such modifications can be generated by techniques with which the skilled worker is familiar, such as “Site Directed Mutagenesis”, “Error Prone PCR”, “DNA-shuffling” (Nature 370, 1994, pp. 389-391) or “Staggered Extension Process” (Nature Biotechnol. 16, 1998, pp. 258-261). The aim of such a modification can be, for example, the insertion of further cleavage sites for restriction enzymes, the removal of DNA in order to truncate the sequence, the substitution of nucleotides to optimize the codons, or the addition of further sequences. Proteins which are encoded via modified nucleic acid sequences must retain the desired function despite a deviating nucleic acid sequence.

The term “functional equivalents” can also relate to the amino acid sequence encoded by the nucleic acid sequence in question. In this case, the term “functional equivalent” describes a protein whose amino acid sequence has a defined percentage of identity or homology with SEQ ID NO:3.

Functional equivalents thus also comprise naturally occurring variants of the herein-described sequences and artificial nucleic acid sequences, for example those which have been obtained by chemical synthesis and which are adapted to the codon usage, and also the amino acid sequences derived from them.

“Genetic control sequence” describes sequences which have an effect on the transcription and, if appropriate, translation of the nucleic acids according to the invention in prokaryotic or eukaryotic organisms. Examples thereof are promoters, terminators or what are known as “enhancer” sequences. In addition to these control sequences, or instead of these sequences, the natural regulation of these sequences may still be present before the actual structural genes and may, if appropriate, have been genetically modified in such a way that the natural regulation has been switched off and the expression of the target gene has been modified, that is to say increased or reduced. The choice of the control sequence depends on the host organism or starting organism. Genetic control sequences furthermore also comprise the 5′-untranslated region, introns or the noncoding 3′-region of genes. Control sequences are furthermore understood as meaning those which make possible homologous recombination or insertion into the genome of a host organism or which permit removal from the genome. Genetic control sequences also comprise further promoters, promoter elements or minimal promoters, and sequences which have an effect on the chromatin structure (for example matrix attachment regions (MARs)), which can modify the expression-governing properties. Thus, genetic control sequences may bring about for example the additional dependence of the tissue-specific expression on certain stress factors. Such elements have been described, for example, for water stress, abscisic acid (Lam E and Chua N H, J Biol Chem 1991; 266(26): 17131-17135), high- and low-temperature stress (Plant Cell 1994, (6): 251-264) and heat stress (Molecular & General Genetics, 1989, 217(2-3): 246-53).

“Homology” between two nucleic acid sequences or polypeptide sequences is defined by the identity of the nucleic acid sequence/polypeptide sequence over in each case the entire sequence length, which is calculated by alignment with the aid of the program algorithm GAP according to Needleman and Wunsch 1970, J. Mol. Biol. 48; 443-453) setting the following parameters for polypeptides:

Gap Weight: 8 Length Weight: 2

Average Match: 2,912 Average Mismatch:−2,003

and the following parameters for nucleic acids:

Gap Weight: 50 Length Weight: 3

Average Match: 10.000 Average Mismatch: 0.000

In the following text, the term identity is also used synonymously with the term “homology”.

“Mutations” of nucleic or amino acid sequences comprise substitutions, additions, deletions, inversions or insertions of one or more nucleotide residues, which may also bring about changes in the corresponding amino acid sequence of the target protein by substitution, insertion or deletion of one or more amino acids, although the functional properties of the target proteins are, overall, essentially retained.

“Natural genetic environment” means the natural chromosomal locus in the organism of origin. In the case of a genomic library, the natural genetic environment of the nucleic acid sequence is preferably retained at least in part. The environment flanks the nucleic acid sequence at least at the 5′- or 3′-side and has a sequence length of at least 50 bp, preferably at least 100 bp, especially preferably at least 500 bp, very especially preferably at least 1000 bp, and most preferably at least 5000 bp.

“Plants” for the purposes of the invention are plant cells, plant tissues, plant organs, or intact plants, such as seeds, tubers, flowers, pollen, fruits, seedlings, roots, leaves, stems or other plant parts. Moreover, the term plants is understood as meaning propagation material such as seeds, fruits, seedlings, slips, tubers, cuttings or root stocks.

“Recombinant DNA” describes a combination of DNA sequences which can be generated by recombinant DNA technology.

“Recombinant DNA technology”: generally known techniques for fusing DNA sequences (for example described in Sambrook et al., 1989, Cold Spring Harbor, N.Y., Cold Spring Harbor Laboratory Press).

“Replication origins” ensure the multiplication of the expression cassettes or vectors according to the invention in microorganisms and yeasts, for example the pBR322 ori or the P15A ori in E. coli (Sambrook et al.: “Molecular Cloning. A Laboratory Manual”, 2nd ed. Cold Spring Harbor Laboratory Press, Cold Spring Harbor, N.Y., 1989) and the ARS1 ori in yeast (Nucleic Acids Research, 2000, 28(10): 2060-2068).

“Reporter genes” encode readily quantifiable proteins. The transformation efficacy or the expression site or timing can be assessed by means of these genes via growth assay, fluorescence assay, chemoluminescence assay, bioluminescence assay or resistance assay or via a photometric measurement (intrinsic color) or enzyme activity. Very especially preferred in this context are reporter proteins (Schenborn E, Groskreutz D. Mol Biotechnol. 1999; 13(1):29-44) such as the “green fluorescent protein” (GFP) (Gerdes H H and Kaether C, FEBS Lett. 1996; 389(1):44-47; Chui W L et al., Curr Biol 1996, 6:325-330; Leffel S M et al., Biotechniques. 23(5):912-8, 1997), chloramphenicol acetyl transferase, a luciferase (Giacomin, Plant Sci 1996, 116:59-72; Scikantha, J Bact 1996, 178:121; Millar et al., Plant Mol Biol Rep 1992 10:324-414), and luciferase genes, in general β-galactosidase or β-glucuronidase (Jefferson et al., EMBO J. 1987, 6, 3901-3907) or the Ura3 gene.

“Selection markers” confer resistance to antibiotics or other toxic compounds: examples which may be mentioned in this context are the neomycin phosphotransferase gene, which confers resistance to the aminoglycoside antibiotics neomycin (G 418), kanamycin, paromycin (Deshayes A et al., EMBO J. 4 (1985) 2731-2737), the sul gene, which encodes a mutated dihydropteroate synthase (Guerineau F et al., Plant Mol. Biol. 1990; 15(1):127-136), the hygromycin B phosphotransferase gene (Gen Bank Accession NO: K 01193) and the shble resistance gene, which confers resistance to the bleomycin antibiotics such as zeocin. Further examples of selection marker genes are genes which confer resistance to 2-deoxyglucose-6-phosphate (WO 98/45456) or phosphinothricin and the like, or those which confer a resistance to antimetabolites, for example the dhfr gene (Reiss, Plant Physiol. (Life Sci. Adv.) 13 (1994) 142-149). Examples of other genes which are suitable are trpB or hisD (Hartman S C and Mulligan R C, Proc Natl Acad Sci USA. 85 (1988) 8047-8051). Another suitable gene is the mannose phosphate isomerase gene (WO 94/20627), the ODC (ornithine decarboxylase) gene (McConlogue, 1987 in: Current Communications in Molecular Biology, Cold Spring Harbor Laboratory, Ed.) or the Aspergillus terreus deaminase (Tamura K et al., Biosci Biotechnol Biochem. 59 (1995) 2336-2338).

“Transformation” describes a process for introducing heterologous DNA into a pro- or eukaryotic cell. The term transformed cell describes not only the product of the transformation process per se, but also all of the transgenic progeny of the transgenic organism generated by the transformation.

“Target/target protein”: a polypeptide encoded via the nucleic acid sequence according to the invention (this term is defined herein below), which may take the form of an enzyme in the traditional sense or, for example, of a structural protein, a protein relevant for developmental processes, regulatory protein such as transcription factors, kinases, phosphatases, receptors, channel subunits, transport proteins, regulatory subunits which confer substrate or activity regulation to an enzyme complex. All of the targets or sites of action share the characteristic that their functional presence is essential for survival or normal development and growth.

“Transgenic”: referring to a nucleic acid sequence, an expression cassette or a vector comprising a nucleic acid sequence according to the invention or an organism transformed with the abovementioned nucleic acid sequence, expression cassette or vector, the term transgenic describes all those constructs which have been generated by genetic engineering methods in which either the nucleic acid sequence of the target protein or a genetic control sequence linked operably to the nucleic acid sequence of the target protein or a combination of the abovementioned possibilities are not in their natural genetic environment or have been modified by recombinant methods. In this context, the modification can be achieved, for example, by mutating one or more nucleotide residues of the nucleic acid sequence in question.

Intracellular protein degradation and its regulation are important for several processes like recycling of aminoacids, prevention of protein agglomeration and regulation of signaling processes (e.g. signalling of phytohormones). Cytosolic Proteins that are to be degraded are ubiquitinylated at the N-terminus and delivered to the proteasome which is established as a complex of a large number of protein components in eucaroytes. Roughly 12% of the genes in Arabidopsis thaliana are encoding proteins envolved in protein degradation by the ubiquitin pathway.

The stroma of plant chloroplasts contains a unique ubiquitin-independent, ATP-dependent protease consisting of two mayor components, a serine-type protease (ClpP) and an ATPase (ClpC, -D, -X) both of which are encoded by enzyme families in Arabidopsis thaliana (for details on the differing nomenclatures in literature see Adam et al. 2001, Plant Physiology 125, pp. 1912-18). Six unique ClpP Isoforms (ClpP1-6) are nuclear encoded in Arabidopsis and at least one ClpP ist encoded in the plastid genome (pClpP) all of which carry the three conserved active site aminoacids characteristical for a catalytic triade of serine proteases. Some sequences of mRNA for putative ATP-dependent protease proteolytic subunits ClpP are disclosed in Nakabayashi et al. (Plant Cell Physiol 40: 504-514, 1999) and Kotani et al. (DNA Research 4, 291-300, 1997). A subunit of Clp protease, which does not show any own activity of a protease is disclosed in WO 2003008440 A. Further Clp gene from algae, tobacco or cyanobacterium are depicted in Huang et al. (Mol. Gen. Genet 244, 151-159, 1994), Shikanai et al. (Plant Cell Physiol. 42, 261-273, 2001) and Clarke et al. (Plant Molecular Biology 37, 791-801, 1998) respectivelly.

Further three nuclear encoded ClpP-Isoforms which miss the conserved amino acid residues of the catalytic triade are found in Arabidopsis (ClpR1, ClpR3, ClpR4). The catalytic activity of ClpR-type ClpP-Isoforms has not been shown so far. At least one ClpP and two ClpX proteins may be targeted to mitochondria in Arabidopsis as deduced from N-terminal signal sequences. ClpP Proteases are conserved in bacteria. The ClpP protease in E. coli was formerly known as “protease Ti”. A knock out of the protease Ti was shown to be not lethal. E. coli ClpP is assambled as a complex of 14 ClpP subunits in two heptameric rings. Co-immunoprecipitation suggests complexes of similar sizes and an ATP-dependet interaction of ClpP and ClpC subunits in Chloroplasts of Arabidopsis thaliana (Halperin et al. 2001, Planta 213, pp. 614-619). Furthermore, a 350 kDa ClpP complex has been identified in Arabidopsis chloroplasts using blue native gel electrophoresis. The complex presumingly containing most of the known ClpP Isoenzymes (Benoit-Peltier et al. 2001, Journal of Biological Chemistry 276, pp. 16348-16327). Consequently the complexity and redundancy of plant Clp proteases is high and detailed information about composition of the clp complex and the functional role of its subunits remain to be clarified. Particularly the role of ClpP redundancy is still unclear.

The ClpP subunit is capable of actively hydrolysing peptides of max. 5 aminoacids in vitro. ClpA,B,C subunits constitute ATP-hydrolysing chaperones which unfold target-proteins and present them for hydrolysis to ClpP (Porankiewicz et al. 1999, Molecular Microbiology 32, 449-458). Involvement of ClpP in the degradation of the cytochrome b6f complex an PSII has been decribed in Chlamydomonas (Majeran et al. 2001, Plant Physiology 23+, pp. 421-433). Functional properties of ClpR-type Clp-Proteases as well as the ClpP like Proeases are yet to be determined.

Surprisingly, it has been found within the scope of the present invention that plants in which a Clp protease was reduced in a selective manner have phenotypes which are comparable with phenotypes generated by herbicide application. Drastic growth retardation and damage such as were observed.

The present invention relates to the use of a polypeptide, which has the activity of nuclear encoded Clp-protease in a method for identifying herbicides, preferably of a polypeptide, which has the activity of nuclear encoded Clp-protease, which is
a) selected from the group consisting of ClpP1-protease, ClpP2-protease, ClpP3-protease, ClpP4-protease and ClpP6-protease; or
b) selected from the group consisting of ClpR1-protease, ClpR3-protease, ClpR4-protease; or
c) ClpP-like-protease, wherein more preferably
a) the ClpP1-protease is encoded by a nucleic acid sequence which comprises:
   i) a nucleic acid sequence with the nucleic acid sequence shown in SEQ ID NO:1, or
   ii) a nucleic acid sequence which, owing to the degeneracy of the genetic code, can be deduced from the amino acid sequence shown in SEQ ID NO:2 by back translating, or
   iii) a functional equivalent of nucleic acid sequence shown in SEQ ID NO:1 which has an identity with SEQ ID NO:1 of has at least 50%; or
   iv) a functional equivalent of the nucleic acid sequence shown in SEQ ID NO:1, which is encoded by an amino acid sequence that has at least an identity of 50% with the SEQ ID NO:2;
b) the ClpP2-protease encoded by a nucleic acid sequence which comprises:
   i) a nucleic acid sequence with the nucleic acid sequence shown in SEQ ID NO:3, or

ii) a nucleic acid sequence which, owing to the degeneracy of the genetic code, can be deduced from the amino acid sequence shown in SEQ ID NO:4 by back translating, or
iii) a functional equivalent of nucleic acid sequence shown in SEQ ID NO:3 which has an identity with SEQ ID NO:3 of has at least 50%; or
iv) a functional equivalent of the nucleic acid sequence shown in SEQ ID NO:3, which is encoded by an amino acid sequence that has at least an identity of 50% with the SEQ ID NO:4;
c) the ClpP3-protease is encoded by a nucleic acid sequence which comprises:
   i) a nucleic acid sequence with the nucleic acid sequence shown in SEQ ID NO:5, or
   ii) a nucleic acid sequence which, owing to the degeneracy of the genetic code, can be deduced from the amino acid sequence shown in SEQ ID NO:6 by back translating, or
   iii) a functional equivalent of nucleic acid sequence shown in SEQ ID NO:5 which has an identity with SEQ ID NO:5 of has at least 50%; or
   iv) a functional equivalent of the nucleic acid sequence shown in SEQ ID NO:5, which is encoded by an amino acid sequence that has at least an identity of 50% with the SEQ ID NO:6;
d) the ClpP4-protease is encoded by a nucleic acid sequence which comprises:
   i) a nucleic acid sequence with the nucleic acid sequence shown in SEQ ID NO:7, or
   ii) a nucleic acid sequence which, owing to the degeneracy of the genetic code, can be deduced from the amino acid sequence shown in SEQ ID NO:8 by back translating, or
   iii) a functional equivalent of nucleic acid sequence shown in SEQ ID NO:7 which has an identity with SEQ ID NO:7 of has at least 50%; or
   iv) a functional equivalent of the nucleic acid sequence shown in SEQ ID NO:7, which is encoded by an amino acid sequence that has at least an identity of 50% with the SEQ ID NO:8;
e) the ClpP6-protease is encoded by a nucleic acid sequence which comprises:
   i) a nucleic acid sequence with the nucleic acid sequence shown in SEQ ID NO:9, or
   ii) a nucleic acid sequence which, owing to the degeneracy of the genetic code, can be deduced from the amino acid sequence shown in SEQ ID NO:10 by back translating, or
   iii) a functional equivalent of nucleic acid sequence shown in SEQ ID NO:9 which has an identity with SEQ ID NO:9 of has at least 50%; or
   iv) a functional equivalent of the nucleic acid sequence shown in SEQ ID NO:9, which is encoded by an amino acid sequence that has at least an identity of 50% with the SEQ ID NO:10;
f) the ClpR1-protease is encoded by a nucleic acid sequence which comprises:
   i) a nucleic acid sequence with the nucleic acid sequence shown in SEQ ID NO:1, or
   ii) a nucleic acid sequence which, owing to the degeneracy of the genetic code, can be deduced from the amino acid sequence shown in SEQ ID NO:12 by back translating, or
   iii) a functional equivalent of nucleic acid sequence shown in SEQ ID NO:11 which has an identity with SEQ ID NO:11 of has at least 50%; or
   iv) a functional equivalent of the nucleic acid sequence shown in SEQ ID NO:11, which is encoded by an amino acid sequence that has at least an identity of 50% with the SEQ ID NO:12;
g) the ClpR3-protease is encoded by a nucleic acid sequence which comprises:
   i) a nucleic acid sequence with the nucleic acid sequence shown in SEQ ID NO:13, or
   ii) a nucleic acid sequence which, owing to the degeneracy of the genetic code, can be deduced from the amino acid sequence shown in SEQ ID NO:14 by back translating, or
   iii) a functional equivalent of nucleic acid sequence shown in SEQ ID NO:13 which has an identity with SEQ ID NO:13 of has at least 50%; or
   iv) a functional equivalent of the nucleic acid sequence shown in SEQ ID NO:13, which is encoded by an amino acid sequence that has at least an identity of 50% with the SEQ ID NO:14;
h) the ClpR4-protease is encoded by a nucleic acid sequence which comprises:
   i) a nucleic acid sequence with the nucleic acid sequence shown in SEQ ID NO:15, or
   ii) a nucleic acid sequence which, owing to the degeneracy of the genetic code, can be deduced from the amino acid sequence shown in SEQ ID NO:16 by back translating, or
   iii) a functional equivalent of nucleic acid sequence shown in SEQ ID NO:15 which has an identity with SEQ ID NO:15 of has at least 50%; or
   iv) a functional equivalent of the nucleic acid sequence shown in SEQ ID NO:15, which is encoded by an amino acid sequence that has at least an identity of 50% with the SEQ ID NO:16;
i) the ClpP like-protease is encoded by a nucleic acid sequence which comprises:
   i) a nucleic acid sequence with the nucleic acid sequence shown in SEQ ID NO:17, or
   ii) a nucleic acid sequence which, owing to the degeneracy of the genetic code, can be deduced from the amino acid sequence shown in SEQ ID NO:18 by back translating, or
   iii) a functional equivalent of nucleic acid sequence shown in SEQ ID NO:17 which has an identity with SEQ ID NO:17 of has at least 50%; or
   iv) a functional equivalent of the nucleic acid sequence shown in SEQ ID NO:17, which is encoded by an amino acid sequence that has at least an identity of 50% with the SEQ ID NO:18;
   wherein the sequences b) i-iv, e) i-iv, f) i-iv and are especially preferred

The term “comprising” in relation to a nucleic acid sequence means that the nucleic acid sequence can be flanked by additional nucleic acid sequences that have on the 5′ end and on the 3′ end or on the 5′ end or on the 3′ end on the end a sequence length of at least 1000 bp, preferably at least 500 bp, more preferably at least 250 bp, most preferably at least 100 bp.

The functional equivalent according to the invention of SEQ ID NO:1 as described in a) iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:1.

The functional equivalents of the nucleic acid sequence SEQ ID NO:1 set forth in a) iv. are encoded by an amino acid sequence, which has the activity of nuclear encoded Clp-protease and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:2.

The functional equivalent according to the invention of SEQ ID NO:3 as described in b) iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:3.

An example of a functional equivalent of SEQ ID NO: 3 is the nucleic acid sequence of Arabidopsis thaliana (Gene Bank Acc. No. AB022327). This sequence is herein incorporated by reference.

The functional equivalents of the nucleic acid sequence set forth SEQ ID NO:3 in b) iv. are encoded by an amino acid sequence, which has the activity of nuclear encoded Clp-protease and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:4.

The functional equivalent according to the invention of SEQ ID NO:5 as described in c) iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:5.

The functional equivalents of the nucleic acid sequence SEQ ID NO:5 set forth in c) iv. are encoded by an amino acid sequence, which has the activity of nuclear encoded Clp-protease and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:6.

The functional equivalent according to the invention of SEQ ID NO:7 as described in d) iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:7.

The functional equivalents of the nucleic acid sequence set forth SEQ ID NO:7 in d) iv. are encoded by an amino acid sequence, which has the activity of nuclear encoded Clp-protease and has at least an identity of 50%, 51%, 52%, 53%, 54%, 55%, 56%, 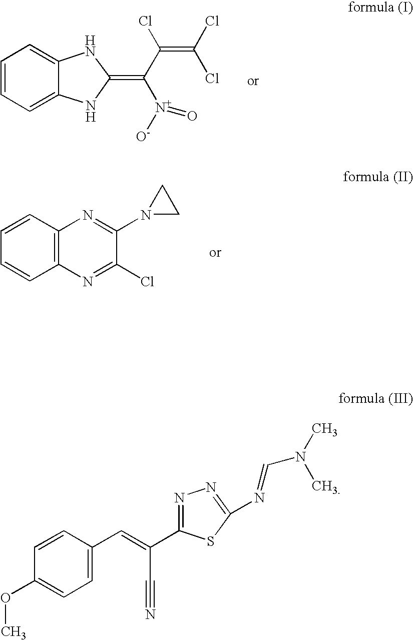 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:7.

The functional equivalent according to the invention of SEQ ID NO:9 as described in e) iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:9.

The functional equivalents of the nucleic acid sequence set forth SEQ ID NO:9 in e) iv. are encoded by an amino acid sequence, which has the activity of nuclear encoded Clp-protease and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:10.

The functional equivalent according to the invention of SEQ ID NO:11 as described in X iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:11.

The functional equivalents of the nucleic acid sequence SEQ ID NO:11 set forth in f) iv. are encoded by an amino acid sequence, which has the activity of nuclear encoded Clp-protease and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:12.

An example of a functional equivalent of SEQ ID NO: 11 is the nucleic acid sequence of Arabidopsis thaliana (Gene Bank Acc. No. AB022330). This sequence is herein incorporated by reference.

The functional equivalent according to the invention of SEQ ID NO:13 as described in g) iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:13.

The functional equivalents of the nucleic acid sequence SEQ ID NO:13 set forth in g) iv. are encoded by an amino acid sequence, which has the activity of nuclear encoded Clp-protease and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:14.

The functional equivalent according to the invention of SEQ ID NO:15 as described in h) iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:15.

The functional equivalents of the nucleic acid sequence SEQ ID NO:15 set forth in h) iv. are encoded by an amino acid sequence, which has the activity of nuclear encoded Clp-protease and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:16.

The functional equivalent according to the invention of SEQ ID NO:17 as described in i) iii), which encodes a polypeptide, which has the activity of nuclear encoded Clp-protease, and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:17.

The functional equivalents of the nucleic acid sequence SEQ ID NO:17 set forth in i) iv. are encoded by an amino acid sequence, which has the activity of nuclear encoded Clp-protease and has at least an identity of 50%, 51%, 52%, 53%, 54%, 55%, 56%, 57%, 58%, 59%, 60%, 61%, 62%, 63%, 64% or 65% or preferably of 66%, 67%, 68%, 69%, 70%, 71%, 72%, 73%, 74%, 75%, 76%, 77%, 78% or 79% more preferably of 80%, 81%, 82%, 83%, 84%, 85%, 86%, 87%, 88%, 89% or 90% most preferably of 91%, 92%, 93%, 94%, 95%, 96%, 97%, 98% or 99% with SEQ ID NO:18.

An example of a functional equivalent of SEQ ID NO:17 is the nucleic acid sequence of Arabidopsis thaliana (Gene Bank Acc. No. AK118525). This sequence is herein incorporated by reference.

Furthermore claimed within the scope of the present invention are plant nucleic acid sequence

I) encoding a ClpP2-protease comprising:

a) a nucleic acid sequence with the nucleic acid sequence shown in SEQ ID NO:3, or
b) a nucleic acid sequence which, owing to the degeneracy of the genetic code, can be deduced from the amino acid sequence shown in SEQ ID NO:4 by backtranslating, or
c) a functional equivalent of nucleic acid sequence shown in SEQ ID NO:1 which has an identity with SEQ ID NO:3 of has at least 66%; or
d) a functional equivalent of the nucleic acid sequence shown in SEQ ID NO:11, which is encoded by an amino acid sequence that has at least an identity of 76% with the SEQ ID NO:4;
   II) encoding a ClpR1-protease comprising:
a) a nucleic acid sequence with the nucleic acid sequence shown in SEQ ID NO:11, or
b) a nucleic acid sequence which, owing to the degeneracy of the genetic code, can be deduced from the amino acid sequence shown in SEQ ID NO:12 by backtranslating, or
c) a functional equivalent of nucleic acid sequence shown in SEQ ID NO:1 which has an identity with SEQ ID NO:11 of has at least 69%; or
d) a functional equivalent of the nucleic acid sequence shown in SEQ ID NO:11, which is encoded by an amino acid sequence that has at least an identity of 71% with the SEQ ID NO:12;
   III) encoding a ClpP-like-protease comprising:
a) a nucleic acid sequence with the nucleic acid sequence shown in SEQ ID NO:17, or
b) a nucleic acid sequence which, owing to the degeneracy of the genetic code, can be deduced from the amino acid sequence shown in SEQ ID NO:18 by backtranslating, or
c) a functional equivalent of nucleic acid sequence shown in SEQ ID NO:1 which has an identity with SEQ ID NO:17 of has at least 67%; or
d) a functional equivalent of the nucleic acid sequence shown in SEQ ID NO:17, which is encoded by an amino acid sequence that has at least an identity of 79% with the SEQ ID NO:18;

The functional equivalent of SEQ ID NO:3 set forth in I c) has at least an identity of 66%, 67%, 68%, 69%, 70%, 71%, 72%, 73%, 74%, by preference at least 75%, 76%, 77%, 78%, 79%, 80%, 81%, 82% or 83%, preferably at least 84%, 85%, 86%, 87%, 88%, 89%, 90%, 91%, 92% or 93%, especially preferably at least 94%, 95%, 96%, 97%, 98% or 99% with SEQ ID NO:3.

The functional equivalents of the nucleic acid sequence SEQ ID NO:3 set forth in 1) d) are encoded by an amino acid sequence, which has the activity of nuclear encoded Clp-protease and has at least an identity of 77%, by preference at least 78%, 79%, 80%, 81%, 82% or 83%, preferably at least 84%, 85%, 86%, 87%, 88%, 89%, 90%, 91%, 92%, 93%, especially preferably at least 94%, 95%, 96%, 97%, 98%, 99% with SEQ ID NO:4.

The functional equivalent of SEQ ID NO:11 set forth in II c) has at least an identity of 69%, 70%, 71%, 72%, 73% or 74%, by preference at least 75%, 76%, 77%, 78%, 79%, 80%, 81%, 82% or 83%, preferably at least 84%, 85%, 86%, 87%, 88%, 89%, 90%, 91%, 92% or 93%, especially preferably at least 94%, 95%, 96%, 97%, 98% or 99% with SEQ ID NO:11.

The functional equivalents of the nucleic acid sequence SEQ ID NO:11 I set forth in II) d) are encoded by an amino acid sequence, which has the activity of nuclear encoded Clp-protease and has at least an identity of 71% by preference at least 72%, 73%, 74%, 75%, 76%, 77%, 78%, 79%, 80%, 81%, 82%, 83%, preferably at least 84%, 85%, 86%, 87%, 88%, 89%, 90%, 91%, 92%, 93%, especially preferably at least 94%, 95%, 96%, 97%, 98%, 99% with SEQ ID NO:12.

The functional equivalent of SEQ ID NO:17 set forth in I c) has at least an identity of 67%, 68%, 69%, 70%, 71%, 72%, 73%, 74%, by preference at least 75%, 76%, 77%, 78%, 79%, 80%, 81%, 82% or 83%, preferably at least 84%, 85%, 86%, 87%, 88%, 89%, 90%, 91%, 92% or 93%, especially preferably at least 94%, 95%, 96%, 97%, 98% or 99% with SEQ ID NO: 17.

The functional equivalents of the nucleic acid sequence SEQ ID NO:17 set forth in I) d) are encoded by an amino acid sequence, which has the activity of nuclear encoded Clp-protease and has at least an identity of 79%, by preference at least 79%, 80%, 81%, 82% or 83%, preferably at least 84%, 85%, 86%, 87%, 88%, 89%, 90%, 91%, 92%, 93%, especially preferably at least 94%, 95%, 96%, 97%, 98%, 99% with SEQ ID NO:18.

The polypeptides encoded by the abovementioned nucleic acid sequences according to I c)-d), II c)-d) and III c)-d) are likewise claimed. The functional equivalents as described in c) and d) are distinguished by the same functionality, i.e. they have the activity of a clp-protease.

The nucleic acid sequences I c)-d), II c)-d) and III c)-d) are hereinbelow termed NCLP-sequences.

The term “nucleic acid sequences according to the invention” which is used hereinbelow refers to nucleic acid sequences encoding a polypeptide, which has the activity of nuclear encoded Clp-protease in a method for identifying herbicides, preferably of a polypeptide, which has the activity of nuclear encoded Clp-protease, which is
a) selected from the group consisting of ClpP1-protease, ClpP2-protease, ClpP3-protease, ClpP4-protease and ClpP6-protease; or
b) selected from the group consisting of ClpR1-protease, ClpR3-protease, ClpR4-protease; or
c) ClpP-like-protease, wherein more preferably
a) the ClpP1-protease is encoded by a nucleic acid sequence which comprises:
   i) a nucleic acid sequence with the nucleic acid sequence shown in SEQ ID NO:1, or
   ii) a nucleic acid sequence which, owing to the degeneracy of the genetic code, can be deduced from the amino acid sequence shown in SEQ ID NO:2 by back translating, or
   iii) a functional equivalent of nucleic acid sequence shown in SEQ ID NO:1 which has an identity with SEQ ID NO:1 of has at least 50%; or
   iv) a functional equivalent of the nucleic acid sequence shown in SEQ ID NO:1, which is encoded by an amino acid sequence that has at least an identity of 50% with the SEQ ID NO:2;
b) the ClpP2-protease encoded by a nucleic acid sequence which comprises:

i) a nucleic acid sequence with the nucleic acid sequence shown in SEQ ID NO:3, or

ii) a nucleic acid sequence which, owing to the degeneracy of the genetic code, can be deduced from the amino acid sequence shown in SEQ ID NO:4 by back translating, or

iii) a functional equivalent of nucleic acid sequence shown in SEQ ID NO:3 which has an identity with SEQ ID NO:3 of has at least 50%; or

iv) a functional equivalent of the nucleic acid sequence shown in SEQ ID NO:3, which is encoded by an amino acid sequence that has at least an identity of 50% with the SEQ ID NO:4;
c) the ClpP3-protease is encoded by a nucleic acid sequence which comprises:

i) a nucleic acid sequence with the nucleic acid sequence shown in SEQ ID NO:5, or

ii) a nucleic acid sequence which, owing to the degeneracy of the genetic code, can be deduced from the amino acid sequence shown in SEQ ID NO:6 by back translating, or

iii) a functional equivalent of nucleic acid sequence shown in SEQ ID NO:5 which has an identity with SEQ ID NO:5 of has at least 50%; or
iv) a functional equivalent of the nucleic acid sequence shown in SEQ ID NO:5, which is encoded by an amino acid sequence that has at least an identity of 50% with the SEQ ID NO:6;
d) the ClpP4-protease is encoded by a nucleic acid sequence which comprises:
   i) a nucleic acid sequence with the nucleic acid sequence shown in SEQ ID NO:7, or
   ii) a nucleic acid sequence which, owing to the degeneracy of the genetic code, can be deduced from the amino acid sequence shown in SEQ ID NO:8 by back translating, or
   iii) a functional equivalent of nucleic acid sequence shown in SEQ ID NO:7 which has an identity with SEQ ID NO:7 of has at least 50%; or
   iv) a functional equivalent of the nucleic acid sequence shown in SEQ ID NO:7, which is encoded by an amino acid sequence that has at least an identity of 50% with the SEQ ID NO:8;
e) the ClpP6-protease is encoded by a nucleic acid sequence which comprises:
   i) a nucleic acid sequence with the nucleic acid sequence shown in SEQ ID NO:9, or
   ii) a nucleic acid sequence which, owing to the degeneracy of the genetic code, can be deduced from the amino acid sequence shown in SEQ ID NO:10 by back translating, or
   iii) a functional equivalent of nucleic acid sequence shown in SEQ ID NO:9 which has an identity with SEQ ID NO:9 of has at least 50%; or
   iv) a functional equivalent of the nucleic acid sequence shown in SEQ ID NO:9, which is encoded by an amino acid sequence that has at least an identity of 50% with the SEQ ID NO:10;
f) the ClpR1-protease is encoded by a nucleic acid sequence which comprises:
   i) a nucleic acid sequence with the nucleic acid sequence shown in SEQ ID NO:11, or
   ii) a nucleic acid sequence which, owing to the degeneracy of the genetic code, can be deduced from the amino acid sequence shown in SEQ ID NO:12 by back translating, or
   iii) a functional equivalent of nucleic acid sequence shown in SEQ ID NO:11 which has an identity with SEQ ID NO:1 of has at least 50%; or
   iv) a functional equivalent of the nucleic acid sequence shown in SEQ ID NO:1, which is encoded by an amino acid sequence that has at least an identity of 50% with the SEQ ID NO:12;
g) the ClpR3-protease is encoded by a nucleic acid sequence which comprises:
   i) a nucleic acid sequence with the nucleic acid sequence shown in SEQ ID NO:13, or
   ii) a nucleic acid sequence which, owing to the degeneracy of the genetic code, can be deduced from the amino acid sequence shown in SEQ ID NO:14 by back translating, or
   iii) a functional equivalent of nucleic acid sequence shown in SEQ ID NO:13 which has an identity with SEQ ID NO:13 of has at least 50%; or
   iv) a functional equivalent of the nucleic acid sequence shown in SEQ ID NO:13, which is encoded by an amino acid sequence that has at least an identity of 50% with the SEQ ID NO:14;
h) the ClpR4-protease is encoded by a nucleic acid sequence which comprises:
   i) a nucleic acid sequence with the nucleic acid sequence shown in SEQ ID NO:15, or
   ii) a nucleic acid sequence which, owing to the degeneracy of the genetic code, can be deduced from the amino acid sequence shown in SEQ ID NO: 16 by back translating, or
   iii) a functional equivalent of nucleic acid sequence shown in SEQ ID NO:15 which has an identity with SEQ ID NO:15 of has at least 50%; or
   iv) a functional equivalent of the nucleic acid sequence shown in SEQ ID NO:15, which is encoded by an amino acid sequence that has at least an identity of 50% with the SEQ ID NO:16;
i) the ClpP like-protease is encoded by a nucleic acid sequence which comprises:
   i) a nucleic acid sequence with the nucleic acid sequence shown in SEQ ID NO:17, or
   ii) a nucleic acid sequence which, owing to the degeneracy of the genetic code, can be deduced from the amino acid sequence shown in SEQ ID NO: 18 by back translating, or
   iii) a functional equivalent of nucleic acid sequence shown in SEQ ID NO:17 which has an identity with SEQ ID NO:17 of has at least 50%; or
   iv) a functional equivalent of the nucleic acid sequence shown in SEQ ID NO:17, which is encoded by an amino acid sequence that has at least an identity of 50% with the SEQ ID NO:18;
   wherein the sequences b) i-iv, e) i-iv, f) i-iv and are especially preferred

A polypeptide, which has the activity of nuclear encoded Clp-protease and is encoded by a nucleic acid sequence according to the invention are hereinbelow simply referred to as “CLP”.

Reduced amounts of glyoxysomal CLP cause growth retardation and necrotic and chlorotic leaves in plants.

The gene products of the nucleic acids according to the invention constitute novel targets for herbicides, which make possible the provision of novel herbicides for controlling undesired plants. Moreover, the gene products of the nucleic acids according to the invention constitute novel targets for growth regulators which make possible the provision of novel growth regulators for regulating the growth of plants.

Undesired plants are understood as meaning, in the broadest sense, all those plants which grow at locations where they are undesired, for example:

Dicotyledonous weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum.

Monocotyledonous weeds from the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristylis, Sagittaria, Eleocharis, Scirpus, Paspalum, Ischaemum, Sphenoclea, Dactyloctenium, Agrostis, Alopecurus, Apera.

SEQ ID NO:1; 3, 5, 7, 9, 11, 13, 15, 17 or 19-21 or parts of SEQ ID NO: 1; 3, 5, 7, 9, 11, 13, 15, 17 or 19-21 can be used for the preparation of hybridization probes. The preparation of these probes and the experimental procedure is known. For example, this can be effected via the selective preparation of radioactive or nonradioactive probes by PCR and the use of suitably labeled oligonucleotides, followed by hybridization experiments. The technologies required for this purpose are detailed, for example, in T. Maniatis, E. F. Fritsch and J. Sambrook, “Molecular Cloning: A Laboratory Manual”, Cold Spring Harbor Laboratory, Cold Spring Harbor, N.Y. (1989). The probes in question can furthermore be modified by standard technologies (Lit. SDM or random mutagenesis) in such a way that they can be employed for further purposes, for example as a probe which hybridizes specifically with mRNA and the corresponding coding sequences in order to analyze the corresponding sequences in other organisms.

The abovementioned probes can be used for the detection and isolation of functional equivalents of SEQ ID NO:2, 4, 6, 8, 10, 12, 14, 16 or 18 from other plant species on the basis of sequence identities. In this context, part or all of the sequence of the SEQ ID NO:2 in question is used as a probe for screening a genomic or cDNA library of the plant species in question or in a computer search for sequences of functional equivalents in electronic databases.

Preferred plant species are the undesired plants which have already been mentioned at the outset.

The invention furthermore relates to expression cassettes comprising
a) genetic control sequences in operable linkage with a NCLP sequence; or
b) additional functional elements, or
c) a combination of a) and b);
   and to the use of expression cassettes comprising
a) genetic control sequences in operable linkage with a nucleic acid sequence according to the invention,
b) additional functional elements, or
c) a combination of a) and b);
   for expressing a CLP, which can be used in in vitro assay systems. Both embodiments of the above-described expression cassettes are referred in the following text as expression cassette according to the invention.

In a preferred embodiment, an expression cassette according to the invention comprises a promoter at the 5′ end of the coding sequence and, at the 3′ end, a transcription termination signal and, if appropriate, further genetic control sequences which are linked operably with the interposed nucleic acid sequence according to the invention.

The expression cassettes according to the invention are also understood as meaning analogs which can be brought about, for example, by a combination of the individual nucleic acid sequences on a polynucleotide (multiple constructs), on a plurality of polynucleotides in a cell (cotransformation) or by sequential transformation.

Advantageous genetic control sequences under point a) for the expression cassettes according to the invention or for vectors comprising expression cassettes according to the invention are, for example, promoters such as the cos, tac, trp, tet, lpp, lac, lacIq, T7, T5, T3, gal, trc, ara, SP6, □-PR or the □-PL promoter, all of which can be used for expressing a CLP, in Gram-negative bacterial strains.

Examples of further advantageous genetic control sequences are present, for example, in the promoters amy and SPO2, both of which can be used for expressing a CLP, in Gram-positive bacterial strains, and in the yeast or fungal promoters AUG1, GPD-1, PX6, TEF, CUP1, PGK, GAP1, TPI, PHO5, AOX1, GAL10/CYC1, CYC1, OliC, ADH, TDH, Kex2, MFA or NMT or combinations of the abovementioned promoters (Degryse et al., Yeast 1995 Jun. 15; 11(7):629-40; Romanos et al. Yeast 1992 June; 8(6):423-88; Benito et al. Eur. J. Plant Pathol. 104, 207-220 (1998); Cregg et al. Biotechnology (N Y) 1993August; 11(8):905-10; Luo X., Gene 1995 Sep. 22; 163(1):127-31: Nacken et al., Gene 1996 Oct. 10; 175(1-2): 253-60; Turgeon et al., Mol Cell Biol 1987 September;7(9):3297-305) or the transcription terminators NMT, Gcyl, TrpC, AOX1, nos, PGK or CYCL (Degryse et al., Yeast 1995 Jun. 15; 11 (7):629-40; Brunelli et al. Yeast 1993 (December 9(12): 1309-18; Frisch et al., Plant Mol. Biol. 27(2), 405409 (1995); Scorer et al., Biotechnology (N.Y. 12 (2), 181-184 (1994), Genbank acc. number Z46232; Zhao et al. Genbank acc number: AF049064; Punt et al., (1987) Gene 56 (1), 117-124), all of which can be used for expressing CLP, in yeast strains.

Examples of genetic control sequences which are suitable for expression in insect cells are the polyhedrin promoter and the p10 promoter (Luckow, V. A. and Summers, M. D. (1988) Bio/Techn. 6, 47-55).

Advantageous genetic control sequences for expressing CLP, in cell culture, in addition to polyadenylation sequences such as, for example, from simian virus 40, are eukaryotic promoters of viral origin such as, for example, promoters of the polyoma virus, adenovirus 2, cytomegalovirus or simian virus 40.

Further advantageous genetic control sequences for expressing nuclear encoded Clp Protease, in plants are present in the plant promoters CaMV/35S [Franck et al., Cell 21(1980) 285-294], PRP1 [Ward et al., Plant. Mol. Biol. 22 (1993)], SSU, OCS, LEB4, USP, STLS1, B33, NOS; FBPaseP (WO 98/18940) or in the ubiquitin or phaseolin promoter; a promoter which is preferably used being, in particular, a plant promoter or a promoter derived from a plant virus. Especially preferred are promoters of viral origin such as the promoter of the cauliflower mosaic virus 35S transcript (Franck et al., Cell 21 (1980), 285-294; Odell et al., Nature 313 (1985), 810-812). Further preferred constitutive promoters are, for example, the agrobacterium nopaline synthase promoter, the TR double promoter, the agrobacterium OCS (octopine synthase) promoter, the ubiquitin promoter, (Holtorf S et al., Plant Mol Biol 1995, 29:637-649), the promoters of the vacuolar ATPase subunits, or the promoter of a proline-rich wheat protein (WO 91/13991).

The expression cassettes may also comprise, as genetic control sequence, a chemically inducible promoter, by which the expression of the exogenous gene in the plant can be controlled at a specific point in time. Such promoters, such as, for example, the PRP1 promoter (Ward et al., Plant. Mol. Biol. 22 (1993), 361-366), a salicylic-acid-inducible promoter (WO 95/19443), a benzenesulfonamide-inducible promoter (EP-A-0388186), a tetracyclin-inducible promoter (Gatz et al., (1992) Plant J. 2, 397404), an abscisic-acid-inducible promoter (EP-A 335528) or an ethanol- or cyclohexanone-inducible promoter (WO 93/21334) may also be used.

Furthermore, suitable promoters are those which confer tissue- or organ-specific expression in, for example, anthers, ovaries, flowers and floral organs, leaves, stomata, trichomes, stems, vascular tissues, roots and seeds. Others which are suitable in addition to the abovementioned constitutive promoters are, in particular, those promoters which ensure leaf-specific expression. Promoters which must be mentioned are the potato cytosolic FBPase promoter (WO 97/05900), the rubisco (ribulose-1,5-bisphosphate carboxylase) SSU (small subunit) promoter or the ST-LSI promoter from potato (Stockhaus et al., EMBO J. 8 (1989), 2445-245). Promoters which are furthermore preferred are those which control expression in seeds and plant embryos. Examples of seed-specific promoters are the phaseolin promoter (U.S. Pat. No. 5,504,200, Bustos M M et al., Plant Cell. 1989; 1(9):839-53), the promoter of the 2S albumin gene (Joseffson L G et al., J Biol Chem 1987, 262:12196-12201), the legumin promoter (Shirsat A et al., Mol Gen Genet. 1989; 215(2):326-331), the USP (unknown seed protein) promoter (Bäumlein H et al., Molecular & General Genetics 1991, 225(3):459-67), the napin gene promoter (Stalberg K, et al., L. Planta 1996, 199:515-519), the sucrose binding protein promoter (WO 00/26388) or the LeB4 promoter (Bäumlein H et al., Mol Gen Genet 1991, 225: 121-128; Fiedler, U. et al., Biotechnology (NY) (1995), 13 (10) 1090).

Further promoters which are suitable as genetic control sequences are, for example, specific promoters for tubers, storage roots or roots, such as, for example, the class I patatin promoter (B33), the potato cathepsin D inhibitor promoter, the starch synthase (GBSS1) promoter or the sporamin promoter, fruit-specific promoters such as, for example, the fruit-specific promoter from tomato (EP-A 409625), fruit-maturation-specific promoters such as, for example, the fruit-maturation-specific promoter from tomato (WO 94/21794), inflorescence-specific promoters such as, for example, the phytoene synthase promoter (WO 92/16635) or the promoter of the P-rr gene (WO 98/22593), or plastid- or chromoplast-specific promoters such as, for example, the RNA polymerase promoter (WO 97/06250), or else the Glycine max phosphoribosyl-pyrophosphate amidotransferase promoter (see also Genbank Accession No. U87999), or another nodespecific promoter as described in EP-A 249676.

Additional functional elements b) are understood as meaning, by way of example but not by limitation, reporter genes, replication origins, selection markers and what are known as affinity tags, in fusion with CLP, directly or by means of a linker optionally comprising a protease cleavage site. Further suitable additional functional elements are sequences which ensure that the product is targeted into the apoplasts, into plastids, the vacuole, the mitochondrion, the peroxisome, the endoplasmatic reticulum (ER) or, owing to the absence of such operative sequences, remains in the compartment where it is formed, the cytosol, (Kermode, Crit. Rev. Plant Sci. 15, 4 (1996), 285-423).

Also in accordance with the invention are vectors comprising at least one copy of the nucleic acid sequences according to the invention and/or the expression cassettes according to the invention.

In addition to plasmids, vectors are furthermore also understood as meaning all of the other known vectors with which the skilled worker is familiar, such as, for example, phages, viruses such as SV40, CMV, baculovirus, adenovirus, transposons, IS elements, phasmids, phagemids, cosmids or linear or circular DNA. These vectors can be replicated autonomously in the host organism or replicated chromosomally; chromosomal replication is preferred.

In a further embodiment of the vector, the nucleic acid construct according to the invention can advantageously also be introduced into the organisms in the form of a linear DNA and integrated into the genome of the host organism via heterologous or homologous recombination. This linear DNA may consist of a linearized plasmid or only of the nucleic acid construct as vector, or the nucleic acid sequences used.

Further prokaryotic or eukaryotic expression systems are mentioned in Chapters 16 and 17 in Sambrook et al., “Molecular Cloning: A Laboratory Manual.” 2nd ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

The expression cassette according to the invention and vectors derived therefrom can be used for transforming bacteria, cyanobacteria, (for example of the genus Synechocystis, Anabaena, Calothrix, Scytonema, Oscillatoria, Plectonema and Nostoc), proteobacteria such as, for example, Magnetococcus sp. MC1, yeasts, filamentous fungi and algae and eukaryoatic nonhuman cells (for example insect cells) with the aim of producing CLP, recombinantly, the generation of a suitable expression cassette depending on the organism in which the gene is to be expressed.

Vectors comprising a NCLP sequence form part of the subject-matter of the present invention.

In a further advantageous embodiment, the nucleic acid sequences according to the invention may also be introduced into an organism by themselves.

If, in addition to the nucleic acid sequences, further genes are to be introduced into the organism, they can all be introduced into the organism together in a single vector, or each individual gene can be introduced into the organism in each case in one vector, it being possible to introduce the different vectors simultaneously or in succession.

In this context, the introduction, into the organisms in question (transformation), of the nucleic acid(s) according to the invention, of the expression cassette or of the vector can be effected in principle by all methods with which the skilled worker is familiar.

In the case of microorganisms, the skilled worker will find suitable methods in the textbooks by Sambrook, J. et al. (1989) “Molecular cloning: A laboratory manual”, Cold Spring Harbor Laboratory Press, von F. M. Ausubel et al. (1994) “Current protocols in molecular biology”, John Wiley and Sons, by D. M. Glover et al., DNA Cloning Vol. 1, (1995), IRL Press (ISBN 019-963476-9), by Kaiser et al. (1994) Methods in Yeast Genetics, Cold Spring Habor Laboratory Press or Guthrie et al. “Guide to Yeast Genetics and Molecular Biology”, Methods in Enzymology, 1994, Academic Press. In the transformation of filamentous fungi, the methods of choice are firstly the generation of protoplasts and transformation with the aid of PEG (Wiebe et al. (1997) Mycol. Res. 101 (7): 971-877; Proctor et al. (1997) Microbiol. 143, 2538-2591), and secondly the transformation with the aid of Agrobacterium tumefaciens (de Groot et al. (1998) Nat. Biotech. 16, 839-842).

In the case of dicots, the methods which have been described for the transformation and regeneration of plants from plant tissues or plant cells can be exploited for transient or stable transformation. Suitable methods are the biolistic method or the transformation of protoplasts (cf., for example, Willmitzer, L., 1993 Transgenic plants. In: Biotechnology, A Multi-Volume Comprehensive Treatise (H. J. Rehm, G. Reed, A. Puhler, P. Stadler, eds.), Vol. 2, 627-659, VCH Weinheim-New York-Basle-Cambridge), electroporation, the incubation of dry embryos in DNA-containing solution, microinjection and the agrobacterium-radiated gene transfer. The above-mentioned methods are described, for example, in B. Jenes et al., Techniques for Gene Transfer, in: Transgenic Plants, Vol. 1, Engineering and Utilization, edited by S. D. Kung and R. Wu, Academic Press (1993) 128-143 and in Potrykus, Annu. Rev. Plant Physiol. Plant Molec. Biol. 42 (1991) 205-225).

The transformation by means of agrobacteria, and the vectors to be used for the transformation, are known to the skilled worker and described extensively in the literature (Bevan et al., Nucl. Acids Res. 12 (1984) 8711. The intermediary vectors can be integrated into the agrobacterial Ti or Ri plasmid by means of homologous recombination owing to sequences which are homologous to sequences in the T-DNA. This plasmid additionally contains the vir region, which is required for the transfer of the T-DNA. Intermediary vectors are not capable of replication in agrobacteria. The intermediary vector can be transferred to Agrobacterium tumefaciens by means of a helper plasmid (conjugation). Binary vectors are capable of replication both in E. coli and in agrobacteria. They contain a selection marker gene and a linker or polylinker which are framed by the right and left T-DNA border region. They can be transformed directly into the agrobacteria (Holsters et al. Mol. Gen. Genet. 163 (1978), 181-187), EP A 0 120 516; Hoekema, in: The Binary Plant Vector System Offsetdrukkerij Kanters B. V., Alblasserdam (1985), Chapter V; Fraley et al., Crit. Rev. Plant. Sci., 4: 1-46 and An et al. EMBO J. 4 (1985), 277-287).

The transformation of monocots by means of vectors based on agrobacterium has also been described (Chan et al., Plant Mol. Biol. 22(1993), 491-506; Hiei et al., Plant J. 6 (1994) 271-282; Deng et al. Science in China 33 (1990), 28-34; Wilmink et al., Plant Cell Reports 11, (1992) 76-80; May et al. Biotechnology 13 (1995) 486-492; Conner and Domisse; Int. J. Plant Sci. 153 (1992) 550-555; Ritchie et al. Transgenic Res. (1993) 252-265). Alternative systems for the transformation of monocots are the transformation by means of biolistic approach (Wan and Lemaux; Plant Physiol. 104 (1994), 37-48; Vasil et al. Biotechnology 11 (1992), 667-674; Ritala et al., Plant Mol. Biol. 24, (1994) 317-325; Spencer et al., Theor. Appl. Genet. 79 (1990), 625-631), protoplast transformation, the electroporation of partially permeabilized cells, and the introduction of DNA by means of glass fibers. In particular the transformation of maize has been described repeatedly in the literature (cf., for example, WO 95/06128; EP 0513849 A1; EP 0465875 A1; EP 0292435 A1; Fromm et al., Biotechnology 8 (1990), 833-844; Gordon-Kamm et al., Plant Cell 2 (1990), 603-618; Koziel et al., Biotechnology 11 (1993) 194-200; Moroc et al., Theor Applied Genetics 80 (190) 721-726).

The successful transformation of other cereal species has also already been described for example in the case of barley (Wan and Lemaux, see above; Ritala et al., see above; wheat (Nehra et al., Plant J. 5(1994) 285-297).

Agrobacteria which have been transformed with a vector according to the invention can likewise be used in a known manner for the transformation of plants, such as test plants like Arabidopsis or crop plants like cereals, maize, oats, rye, barley, wheat, soya, rice, cotton, sugarbeet, canola, sunflower, flax, hemp, potato, tobacco, tomato, carrot, capsicum, oilseed rape, tapioca, cassaya, arrowroot, Tagetes, alfalfa, lettuce and the various tree, nut and grapevine species, for example by bathing scarified leaves or leaf segments in an agrobacterial solution and subsequently growing them in suitable media.

The genetically modified plant cells can be regenerated via all methods with which the skilled worker is familiar. Such methods can be found in the abovementioned publications by S. D. Kung and R. Wu, Potrykus or Höfgen and Willmitzer.

The transgenic organisms generated by transformation with one of the above-described embodiments of an expression cassette comprising a nucleic acid sequence according to the invention or a vector comprising the abovementioned expression cassette, and the recombinant CLP, which can be obtained from the transgenic organism by means of expression, form part of the subject matter of the present invention. The use of transgenic organisms comprising an expression cassette according to the invention, for example for providing recombinant protein, and/or the use of these organisms in in-vivo assay systems likewise form part of the subject matter of the present invention.

Preferred organisms for the recombinant expression are not only bacteria, yeasts, mosses, algae and fungi, but also eukaryotic cell lines.

Preferred mosses are Physcomitrella patens or other mosses described in Kryptogamen [Cryptogamia], Vol. 2, Moose, Farne [Mosses, Ferns], 1991, Springer Verlag (ISBN 3540536515).

Preferred within the bacteria are, for example, bacteria from the genus Escherichia, Erwinia, Flavobacterium, Alcaligenes or cyanobacteria, for example from the genus Synechocystis, Anabaena, Calothrix, Scytonema, Oscillatoria, Plectonema and Nostoc, especially preferably Synechocystis or Anabaena.

Preferred yeasts are Candida, Saccharomyces, Schizosaccheromyces, Hansenula or Pichia.

Preferred fungi are Aspergillus, Trichoderma, Ashbya, Neurospora, Fusarium, Beauveria, Mortierella, Saprolegnia, Pythium, or other fungi described in Indian Chem Engr. Section B. Vol 37, No 1,2 (1995).

Preferred plants are selected in particular among monocotyledonous crop plants such as, for example, cereal species such as wheat, barley, sorghum or millet, rye, triticale, maize, rice or oats, and sugarcane. The transgenic plants according to the invention are, furthermore, in particular selected from among dicotyledonous crop plants such as, for example, Brassicaceae such as oilseed rape, cress, Arabidopsis, cabbages or canola; Leguminosae such as soyabean, alfalfa, pea, beans or peanut, Solanaceae such as potato, tobacco, tomato, egg plant or capsicum; Asteraceae such as sunflower, Tagetes, lettuce or Calendula; Cucurbitaceae such as melon, pumpkin/squash or zucchini, or linseed, cotton, hemp, flax, red pepper, carrot, sugar beet, or various tree, nut and grapevine species.

In principle, transgenic animals such as, for example, C. elegans, are also suitable as host organisms.

Also preferred is the use of expression systems and vectors which are available to the public or commercially available.

Those which must be mentioned for use in E. coli bacteria are the typical advantageous commercially available fusion and expression vectors pGEX [Pharmacia Biotech Inc; Smith, D. B. and Johnson, K. S. (1988) Gene 67:31-40], pMAL (New England Biolabs, Beverly, Mass.) and pRIT5 (Pharmacia, Piscataway, N.J.), which contains glutathione S transferase (GST), maltose binding protein or protein A, the pTrc vectors (Amann et al., (1988) Gene 69:301-315), “pKK233-2” from CLONTECH, Palo Alto, Calif. and the “pET”, and the “pBAD” vector series from Stratagene, La Jolla and the TOPO-TA vector series drom Invitrogen.

Further advantageous vectors for use in yeast are pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES derivatives, pGAPZ derivatives, pPICZ derivatives, and the vectors of the “Pichia Expression Kit” (Invitrogen Corporation, San Diego, Calif.). Vectors for use in filamentous fungi are described in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge.

As an alternative, insect cell expression vectors may also be used advantageously, for example for expression in Sf9, Sf21 or Hi5 cells, which are infected via recombinant Baculoviruses. Examples of these are the vectors of the pAc series (Smith et al. (1983) Mol. Cell. Biol. 3:2156-2165) and the pVL series (Lucklow and Summers (1989) Virology 170:31-39). Others which may be mentioned are the Baculovirus expression systems “MaxBac 2.0 Kit” and “Insect Select System” from Invitrogen, Carlsbad or “BacPAK Baculovirus Expression System” from CLONTECH, Palo Alto, Calif. Insect cells are particularly suitable for overexpressing eukaryotic proteins since they effect posttranslational modifications of the proteins which are not possible in bacteria and yeasts. The skilled worker is familiar with the handling of cultured insect cells and with their infection for expressing proteins, which can be carried out analogously to known methods (Luckow and Summers, Bio/Tech. 6, 1988, pp. 47-55; Glover and Hames (eds) in DNA Cloning 2, A practical Approach, Expression Systems, Second Edition, Oxford University Press, 1995, 205-244).

Plant cells or algal cells are others which can be used advantageously for expressing genes. Examples of plant expression vectors can be found as mentioned above in Becker, D., et al. (1992) “New plant binary vectors with selectable markers located proximal to the left border”, Plant Mol. Biol. 20: 1195-1197 or in Bevan, M. W. (1984) “Binary Agrobacterium vectors for plant transformation”, Nucl. Acid. Res. 12: 8711-8721.

Moreover, the nucleic acid sequences according to the invention can be expressed in mammalian cells. Examples of suitable expression vectors are pCDM8 and pMT2PC, which are mentioned in: Seed, B. (1987) Nature 329:840 or Kaufman et al. (1987) EMBO J. 6:187-195). Promoters preferably to be used in this context are of viral origin such as, for example, promoters of polyoma virus, adenovirus 2, cytomegalovirus or simian virus 40. Further prokaryotic and eukaryotic expression systems are mentioned in Chapter 16 and 17 in Sambrook et al., Molecular Cloning: A Laboratory Manual. 2nd ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

The transgenic organisms which comprise a NCLP sequence are claimed within the scope of the present invention.

All of the above-described embodiments of the transgenic organisms, which comprise at least one nucleic acid sequence according to the invention come under the term “transgenic organism according to the invention”.

The present invention furthermore relates to the use of CLP, in a method for identifying herbicidally active test compounds.

The method according to the invention for identifying herbicidally active compounds preferably comprises the following steps:
i. bringing CLP into contact with one or more test compounds under conditions which permit the test compound(s) to bind to a nucleic acid sequence according to the invention or to CLP, and
ii. detecting whether the test compound binds to the CLP of i), or
iii. detecting whether the test compound reduces or blocks the enzymatic or biological activity of CLP of i), or
iv. detecting whether the test compound reduces or blocks the transcription, translation or expression of CLP of i).

The detection in accordance with step (ii) of the above method can be effected using techniques which identify the interaction between the polypeptide and ligand. In this context, either the test compound or the enzyme can contain a detectable label such as, for example, a fluorescent label, a radioisotope, a chemiluminescent label or an enzyme label. Examples of enzyme labels are horseradish peroxidase, alkaline phosphatase or luciferase. The subsequent detection depends on the label and is known to the skilled worker.

In this context, five preferred embodiments which are also suitable for high-throughput methods (HTS) in connection with the present invention must be mentioned in particular:
1. The average diffusion rate of a fluorescent molecule as a function of the mass can be determined in a small sample volume via fluorescence correlation spectroscopy (FCS) (Proc. Natl. Acad. Sci. USA (1994) 11753-11575). FCS can be employed for determining protein/ligand interactions by measuring the change in the mass, or the changed diffusion rate which this entails, of a test compound when binding to CLP. A method according to the invention can be designed directly for measuring the binding of a test compound labeled by a fluorescent molecule. As an alternative, the method according to the invention can be designed in such a way that a chemical reference compound which is labeled by a fluorescent molecule is displaced by further test compounds (“displacement assay”).
2. Fluoresence polarization exploits the characteristic of a quiescent fluorophore excited with polarized light to likewise emit polarized light. If, however, the fluorophore is allowed to rotate during the excited state, the polarization of the fluorescent light which is emitted is more or less lost. Under otherwise identical conditions (for example temperature, viscosity, solvent), the rotation is a function of molecule size, whereby findings regarding the size of the fluorophore-bound residue can be obtained via the reading (Methods in Enzymology 246 (1995), pp. 283-300). A method according to the invention can be designed directly for measuring the binding of a test compound labeled with a fluorescent molecule to the CLP. As an alternative, the method according to the invention may also take the form of the “displacement assay” described under 1.
3. Fluorescence resonance energy transfer (FRET) is based on the irradiation-free energy transfer between two spatially adjacent fluorescent molecules under suitable conditions. A prerequisite is that the emission spectrum of the donor molecule overlaps with the excitation spectrum of the acceptor molecule. The fluorescent label of CLP, and binding test compound, the binding can be measured by means of FRET (Cytometry 34, 1998, pp. 159-179). As an alternative, the method according to the invention may also take the form of the “displacement assay” described under 1. An especially suitable embodiment of FRET technology is “Homogeneous Time Resolved Fluorescence” (HTRF) as can be obtained from Packard BioScience.
4. Surface-enhanced laser desorption/ionization (SELDI) in combination with a time-of-flight mass spectrometer (MALDI-TOF) makes possible the rapid analysis of molecules on a support and can be used for analyzing protein/ligand interactions (Worral et al., (1998) Anal. Biochem. 70:750-756). In a preferred embodiment, CLP, is immobilized on a suitable support and incubated with the test compound. After one or more suitable wash steps, the test compound molecules which are additionally bound to CLP, can be detected by means of the above-mentioned methodology and test compounds which are bound to CLP, can thus be selected.
5. The measurement of surface plasmon resonance is based on the change in the refractive index at a surface when a test compound binds to a protein which is immobilized to said surface. Since the change in the refractive index is identical for virtually all proteins and polypeptides for a defined change in the mass concentration at the surface, this method can be applied to any protein in principle (Lindberg et al. Sensor Actuators 4 (1983) 299-304; Malmquist Nature 361 (1993) 186-187). The measurement can be carried out for example with the automatic analyzer based on surface plasmon resonance which is available from Biacore (Freiburg) at a throughput of, currently, up to 384 samples per day. A method according to the invention can be designed directly for measuring the binding of a test compound to CLP. As an alternative, the method according to the invention may also take the form of the “displacement assay” described under 1.

The compounds identified via the abovementioned methods 1 to 5 may be suitable as inhibitors. All of the substances identified via the abovementioned methods can subsequently be checked for their herbicidal action in another embodiment of the method according to the invention.

Furthermore, there exists the possibility of detecting further candidates for herbicidal active ingredients by molecular modeling via elucidation of the three-dimensional structure of CLP, by x-ray structure analysis. The preparation of protein crystals required for x-ray structure analysis, and the relevant measurements and subsequent evaluations of these measurements, the detection of a binding site in the protein, and the prediction of potential inhibitor structures are known to the skilled worker. In principle, an optimization of the compound identified by the abovementioned methods is also possible via molecular modeling.

A preferred embodiment of the method according to the invention, which is based on steps i) and ii), consists in selecting a test compound which reduces or blocks the activity of the CLP. Preferably, the activity of the CLP, incubated with the test compound is herein compared with the activity of a CLP, not incubated with a test compound.

A more preferred embodiment of the method based on steps i) and ii) consists in
i. expressing CLP in a transgenic organism according to the invention or growing an organism which naturally contains a CLP,
ii. bringing CLP, of step i) in the cell digest of the transgenic or nontransgenic organism, in partially purified or in homogeneously purified form, into contact with a test compound; and
iii. selecting a compound which reduces or blocks the activity of the nuclear encoded Clp Protease. Preferably the activity of CLP incubated with the test compound is herein compared with the activity of a CLP, not incubated with a test compound.

The solution containing the CLP, can consist of the lysate of the original organism or of the transgenic organism which has been transformed with an expression cassette according to the invention. If necessary, the CLP, can be purified partially or fully via customary methods. A general overview over current protein purification techniques is described, for example, in Ausubel, F. M. et al., Current Protocols in Molecular Biology, Greene Publishing Assoc. and Wiley-Interscience (1994); ISBN 0-87969-309-6. In the case of recombinant preparation, the protein which has been fused with an affinity tag can be purified via affinity chromatography as is known to the skilled worker.

The CLP, which is required for in vitro methods can thus be isolated either by means of heterologous expression from a transgenic organism according to the invention or from an organism containing CLP, for example from an undesired plant, the term “undesired plant” being understood as meaning the species mentioned at the outset.

To identify herbicidal compounds, the CLP, is now incubated with a test compound. After a reaction time, the enzymatic activity of the CLP, incubated with the test compound is determined in comparison with a CLP, not incubated with a test compound. If the CLP, is inhibited, a significant decrease in activity in comparison with the activity of the noninhibited polypeptide according to the invention is observed, the result being a reduction of at least 10%, advantageously at least 20%, preferably at least 30%, especially preferably by at least 50%, up to 100% reduction (blocking). Preferred is an inhibition of at least 50% at test compound concentrations of 10⁻⁴M, preferably at 10⁻⁵M, especially preferably of 10⁻⁶M, based on enzyme concentration in the micromolar range.

The enzymatic activity of CLP, can be determined for example by an activity assay in which the increase of the product, the decrease of the substrate (or starting material) or the decrease or increase of the cofactor are determined, or by a combination of at least two of the abovementioned parameters, as a function of a defined period of time.

Examples of suitable substrates are, for example small peptides and modified small peptides like peptides coupled to a fluorogenic molecule such as aminomethylcoumarin and succinylated peptides.

If appropriate, derivatives of the abovementioned compounds which contain a detectable label such as, for example, a fluorescent label (e.g. fluorogenic substrates such as N-Suc-Leu-Tyr-(7-amino-4-methylcoumarine) (SLT-AMC), Suc-Ala-Ala-Ala-AMC, Suc-Leu-Leu-Val-Tyr-AMC, Suc-Ala-Ala-Phe-AMC, Suc-Ile-Ile-Trp-AMC, Suc-Ala-Phe-LysAMC), a radioisotope label or a chemiluminescent label, may also be used.

The amounts of substrates to be employed in the activity tests may range between 0.5 and 100 mM, based on 1-100 ug/ml enzyme. [

The activity can be determined for example by tracking Proteolysis fluorimetrically when using fluorogenic Peptide substrates analogously to the method described by Woo et al. 1989 The Journal of Biological Chemistry 264, pp. 2088-2091, which is herein incorporated by reference.

The activity may also be determined in an ATP-dependent fashion in the presence of ClpA, ClpB or ClpC Protein as described in Halperin et al. 2001, Planta 213, pp. 614-619. The preferred Substrate is then b-casein.

Furthermore the activity may be measured by HPLC and HPLC-MS methods detecting fragments of the peptides used as substrates.

Another preferred embodiment of the method according to the invention which is based on steps i) and iii) consists of the following steps:
i. generating a transgenic organism according to the invention comprising a nucleic acid sequence according to the invention, wherein CLP is expressed recombinantly;
ii. applying a test compound to the transgenic organism of i) and to a nontransgenic organism of the same species;
iii. determining the growth or the viability of the transgenic and the nontransgenic organisms after application of the test substance, and
iv. selecting test compounds which bring about a reduced growth or a limited viability of the nontransgenic organism in comparison with the growth of the transgenic organism.

In this context, the difference in growth in step iv) for the selection of a herbicidally active inhibitor amounts to at least 10%, by preference 20%, preferably 30%, especially preferably 40% and very especially preferably 50%.

The transgenic organism in this context is preferably a plant, an alga, a cyanobacterium, for example of the genus Synechocystis or a proteobacterium such as, for example, Magnetococcus sp. MC1, preferably plants which can be transformed by means of customary techniques, such as Arabidopsis thaliana Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays, or cyanobacteria which can be transformed readily, such as Synechocystis, into which the sequence encoding a polypeptide according to the invention has been incorporated by transformation. These transgenic organisms thus show increased tolerance to compounds which inhibit the polypeptide according to the invention. “Knock-out” mutants in which the analogous CLProtease gene which is naturally present in this organism has been selectively switched off may also be used.

However, the abovementioned embodiment of the method according to the invention can also be used for identifying substances with a growth-regulatory action. In this context, the transgenic organism employed is a plant. The method for identifying substances with growth-regulatory activity thus comprises the following steps:
i. generating a transgenic plant comprising a nucleic acid sequence according to the invention encoding CLP, wherein CLP is expressed recombinantly;
ii. applying a test substance to the transgenic plant of i) and to a nontransgenic plant of the same variety,
iii. determining the growth or the viability of the transgenic plant and the nontransgenic plant after application of the test compound, and
iv. selecting test substances which bring about a reduced growth of the nontransgenic plant in comparison with the growth of the transgenic plant.

Here, step iv) involves the selection of test compounds which bring about a modified growth of the nontransgenic organism in comparison with the growth of the transgenic organism. Modified growth is understood as meaning, in this context, inhibition of the vegetative growth of the plants, which can manifest itself in particular in reduced longitudinal growth. Accordingly, the treated plants show stunted growth; moreover, their leaves are darker in color. In addition, modified growth is also understood as meaning a change in the course of maturation over time, the inhibition or promotion of lateral branched growth of the plants, shortened or extended developmental stages, increased standing ability, the growth of larger amounts of buds, flowers, leaves, fruits, seed kernels, roots and tubers, an increased sugar content in plants such as sugarbeet, sugar cane and citrus fruit, an increased protein content in plants such as cereals or soybean, or stimulation of the latex flow in rubber trees. The skilled worker is familiar with the detection of such modified growth.

It is also possible, in the method according to the invention, to employ a plurality of test compounds in a method according to the invention. If a group of test compounds affect the target, then it is either possible directly to isolate the individual test compounds or to divide the group of test compounds into a variety of subgroups, for example when it consists of a multiplicity of different components, in order to thus reduce the number of the different test compounds in the method according to the invention. The method according to the invention is then repeated with the individual test compound or the relevant subgroup of test compounds. Depending on the complexity of the sample, the above-described steps can be carried out repeatedly, preferably until the subgroup identified in accordance with the method according to the invention only comprises a small number of test compounds, or indeed just one test compound.

All of the above-described methods for identifying inhibitors with herbicidal or growth-regulatory activity are hereinbelow referred to as “methods according to the invention”.

All of the compounds which have been identified via the methods according to the invention can subsequently be tested in vivo for their herbicidal and growth-regulatory activity. One possibility of testing the compounds for herbicidal action is to use duckweed, Lemna minor, in microtiter plates. Parameters which can be measured are changes in the chlorophyll content and the photosynthesis rate. It is also possible to apply the compound directly to undesired plants, it being possible to identify the herbicidal action for example via restricted growth.

The method according to the invention can advantageously also be carried out in high-throughput methods, known as HTS, which makes possible the simultaneous testing of a multiplicity of different compounds.

The use of supports which contain one or more of the nucleic acid molecules according to the invention, one or more of the vectors containing the nucleic acid sequence according to the invention, one or more transgenic organisms containing at least one of the nucleic acid sequences according to the invention or one or more (poly)peptides encoded via the nucleic acid sequences according to the invention lends itself to carrying out HTS in practice.

Supports which contain one or more of the NCLP sequences, one or more of the vectors comprising the NCLP sequences one or more transgenic organisms containing at least one NCLP sequences or one or more (poly)peptides encoded by the NCLP sequences are part of the present invention.

The support used can be solid or liquid, but is preferably solid and especially preferably a microtiter plate. The abovementioned supports also form part of the subject matter of the present invention. In accordance with the most widely used technique, 96-well, 384-well and 1536-well microtiter plates which, as a rule, can comprise volumes of 200 □l, are used. Besides the microtiter plates, the further components of an HTS system which match the corresponding microtiter plates, such as a large number of instruments, materials, automatic pipetting devices, robots, automated plate readers and plate washers, are commercially available.

In addition to the HTS systems based on microtiter plates, what are known as “free-format assays” or assay systems where no physical barriers exist between the samples, as described, for example, in Jayaickreme et al., Proc. Natl. Acad. Sci U.S.A. 19 (1994) 161418; Chelsky, “Strategies for Screening Combinatorial Libraries”, First Annual Conference of The Society for Biomolecular Screening in Philadelphia, Pa. (Nov. 710, 1995); Salmon et al., Molecular Diversity 2 (1996), 5763 and U.S. Pat. No. 5,976,813, may also be used.

The invention furthermore relates to herbicidally active compounds identified by the methods according to the invention. These compounds are hereinbelow referred to as “selected compounds”. They have a molecular weight of less than 1000 g/mol, advantageously less than 500 g/mol, preferably less than 400 g/mol, especially preferably less than 300 g/mol. Herbicidally active compounds have a Ki value of less than 1 mM, preferably less than 1 μM, especially preferably less than 0.1 μM, very especially preferably less than 0.01 μM.
Examples for Herbicidally Active Compounds Identified with the Above Mentioned HTS Methods are the Compounds of the Formula:

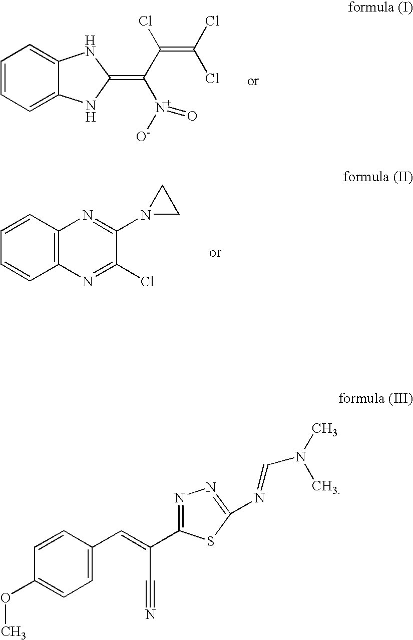

The invention furthermore relates to compounds with growth-regulatory activity identified by the methods according to the invention. These compounds too are hereinbelow referred to as “selected compounds”.

Naturally, the selected compounds can also be present in the form of their agriculturally useful salts. Agriculturally useful salts which are suitable are mainly the salts of those cations, or the acid addition salts of those acids, whose cations, or anions, do not adversely affect the herbicidal action of the herbicidally active compounds identified via the methods according to the invention.

If the selected compounds contain asymmetrically substituted □-carbon atoms, they may furthermore also be present in the form of racemates, enantiomer mixtures, pure enantiomers or, if they have chiral substituents, also in the form of diastereomer mixtures.

The selected compounds can be chemically synthesized substances or substances produced by microbes and can be found, for example, in cell extracts of, for example, plants, animals or microorganisms. The reaction mixture can be a cell-free extract or comprise a cell or cell culture. Suitable methods are known to the skilled worker and are described generally for example in Alberts, Molecular Biology the cell, 3rd Edition (1994), for example chapter 17. The selected compounds may also originate from comprehensive substance libraries.

Candidate test compounds can be expression libraries such as, for example, cDNA expression libraries, peptides, proteins, nucleic acids, antibodies, small organic substances, hormones, PNAs or the like (Milner, Nature Medicin 1 (1995), 879-880; Hupp, Cell. 83 (1995), 237-245; Gibbs, Cell. 79 (1994), 193-198 and references cited therein).

The selected compounds can be used for controlling undesired vegetation and/or as growth regulators. Herbicidal compositions comprising the selected compounds afford very good control of vegetation on noncrop areas. In crops such as wheat, rice, maize, soybean and cotton, they act against broad-leaved weeds and grass weeds without inflicting any significant damage on the crop plants. This effect is observed in particular at low application rates. The selected compounds can be used for controlling the harmful plants which have already been mentioned above.

Depending on the application method in question, selected compounds, or herbicidal compositions comprising them, can advantageously also be employed in a further number of crop plants for eliminating undesired plants. Examples of suitable crops are:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays.

In addition, the selected compounds can also be used in crops which tolerate the action of herbicides owing to breeding, including recombinant methods. The generation of such crops is described hereinbelow.

The invention furthermore relates to a method of preparing the herbicidal or growth-regulatory composition which has already been mentioned above, which comprises formulating selected compounds with suitable auxiliaries to give crop protection products.

The selected compounds can be formulated for example in the form of directly sprayable aqueous solutions, powders, suspensions, also highly concentrated aqueous, oily or other suspensions or suspoemulsions or dispersions, emulsifiable concentrates, emulsions, oil dispersions, pastes, dusts, materials for spreading or granules, and applied by means of spraying, atomizing, dusting, spreading or pouring. The use forms depend on the intended use and the nature of the selected compounds; in any case, they should guarantee the finest possible distribution of the selected compounds. The herbicidal compositions comprise a herbicidally active amount of at least one selected compound and auxiliaries conventionally used in the formulation of herbicidal compositions.

For the preparation of emulsions, pastes or aqueous or oily formulations and dispersible concentrates (DC), the selected compounds can be dissolved or dispersed in an oil or solvent, it being possible to add further formulation auxiliaries for homogenization. However, it is also possible to prepare liquid or solid concentrates from selected compound, if appropriate solvents or oil and, optionally, further auxiliaries comprising liquid or solid concentrates, and these concentrates are suitable for dilution with water. The following can be mentioned: emulsifiable concentrates (EC, EW), suspensions (SC), soluble concentrates (SL), dispersible concentrates (DC), pastes, pills, wettable powders or granules, it being possible for the solid formulations either to be soluble or dispersible (wettable) in water. In addition, suitable powders or granules or tablets can additionally be provided with a solid coating which prevents abrasion or premature release of the active ingredient.

In principle, the term “auxiliaries” is understood as meaning the following classes of compounds: antifoams, thickeners, wetting agents, tackifiers, dispersants, emulsifiers, bactericides and/or thixotropic agents. The skilled worker is familiar with the meaning of the abovementioned agents.

SLs, EWs and ECs can be prepared by simply mixing the ingredients in question; powders can be prepared by mixing or grinding in specific types of mills (for example hammer mills). DCs, SCs and SEs are usually prepared by wet milling, it being possible to prepare an SE from an SC by addition of an organic phase which may comprise further auxiliaries or selected compounds. The preparation is known. Powders, materials for spreading and dusts can advantageously be prepared by mixing or cogrinding the active substances together with a solid carrier. Granules, for example coated granules, impregnated granules and homogeneous granules, can be prepared by binding the selected compounds to solid carriers. The skilled worker is familiar with further details regarding their preparation, which are mentioned for example in the following publications: U.S. Pat. No. 3,060,084, EP-A 707445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Federal Republic of Germany), 2001.

The skilled worker is familiar with a multiplicity of inert liquid and/or solid carriers which are suitable for the formulations according to the invention, such as, for example, liquid additives such as mineral oil fractions of medium to high boiling point such as kerosene or diesel oil, furthermore coal tar oils and oils of vegetable or animal origin, aliphatic, cyclic and aromatic hydrocarbons, for example paraffin, tetrahydrophthalene, alkylated naphthalenes or their derivatives, alkylated benzenes or their derivatives, alcohols such as methanol, ethanol, propanol, butanol and cyclohexanol, ketones such as cyclohexanone, or strongly polar solvents, for example amines such as N-methylpyrrolidone or water.

Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and products of vegetable origin such as cereal meal, tree bark meal, wood meal and nutshell meal, cellulose powders or other solid carriers.

The skilled worker is familiar with the multiplicity of surface-active substances (surfactants) which are suitable for the formulations according to the invention such as, for example, alkali metal salts, alkaline earth metal salts or ammonium salts of aromatic sulfonic acids for example lignosulfonic acid, phenolsulfonic acid, naphthalenesulfonic acid, and dibutyinaphthalenesulfonic acid, and of fatty acids, of alkyl- and alkylarylsulfonates, of alkyl sulfates, lauryl ether sulfates and fatty alcohol sulfates, and salts of sulfated hexa-, hepta- and octadecanols and of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl polyglycol ethers, tributylphenyl polyglycol ether, alkylaryl polyether alcohols, isotridecyl alcohol, fatty alcohol/ethylene oxide condensates, ethoxylated caster oil, polyoxyethylene alkyl ethers or polyoxypropylene alkyl ethers, lauryl alcohol polyglycol ether acetate, sorbitol esters, lignosulfite waste liquors or methylcellulose.

The herbicidal compositions, or the selected compounds, can be applied pre- or post-emergence. If the selected compounds are less well tolerated by certain crop plants, application techniques may be used in which the selected compounds are sprayed, with the aid of the spraying apparatus, in such a way that they come into as little contact, if any, with the leaves of the sensitive crop plants while the selected compounds reach the leaves of undesired plants which grow underneath, or the bare soil surface (post-directed, lay-by).

Depending on the intended purpose of the control measures, the season, the target plants and the growth stage, the application rates of selected compounds amount to 0.001 to 3.0, preferably 0.01 to 1.0 kg/ha.

The invention is illustrated in greater detail by the examples which follow, which are not to be considered as limiting.

General DNA Manipulation and Cloning Methods

Cloning methods such as, for example, restriction cleavages, agarose gel electrophoreses, purification of DNA fragments, transfer of nucleic acids to nitrocellulose and nylon membranes, linking DNA fragments, transformation of Escherichia coli cells, growing bacterium and sequence analyses of recombinant DNA were carried out as described by Sambrook et al. (1989) (Cold Spring Harbor Laboratory Press: ISBN 0-87969-309-6) and Ausubel, F. M. et al., Current Protocols in Molecular Biology, Greene Publishing Assoc. and Wiley-Interscience (1994); ISBN 0-87969-309-6.

Molecular-biological standard methods for plants and plant transformation methods are described in Schultz et al., Plant Molecular Biology Manual, Kluwer Academic Publishers (1998), Reither et al., Methods in Arabidopsis Research, World scientific press (1992) and Arabidopsis: A Laboratory Manual (2001), ISBN 0-87969-573-0.

The bacterial strains used hereinbelow (E. coli DH5, XL-1 blue) were obtained from Stratagene, BRL Gibco or Invitrogen, Carlsberg, Calif. The vectors used for cloning were pUC 19 from Amersham Pharmacia (Freiburg) and the vector pBinAR (Hofgen and Willmitzer, Plant Science 66, 1990, 221-230).

### EXAMPLE 1

### Generation of a cDNA Library in the Plant Transformation Vector

To generate a cDNA library (hereinbelow termed “binary cDNA library”) in a vector which can be used directly for transforming plants, mRNA was isolated from a variety of plant tissues and transcribed into double-stranded cDNA using the cDNA Synthese Kit (Amersham Pharmacia Biotech, Freiburg). The cDNA first-strand synthesis was carried out using T12-18 oligonucleotides following the manufacturer's instructions. After size fractionation and the ligation of EcoRI-NotI adapters following the manufacturer's instructions and filling up the overhangs with Pfu DNA polymerase (Stratagene), the cDNA population was normalized. The method of Kohci et al, 1995, Plant Journal 8, 771-776 was followed, the cDNA being amplified by PCR with the oligonucleotide N1 under the conditions given in Table 1.

TABLE 1

| Temperature [° C.] | Time [sec] | Number of cycles |
|---|---|---|
| 94 | 300 | 1 |
| 94 | 8 | 10 |
| 52 | 60 | |
| 72 | 180 | |
| 94 | 8 | 10 |
| 50 | 60 | |
| 72 | 180 | |
| 94 | 8 | 10 |
| 48 | 60 | |
| 72 | 180 | |
| 72 | 420 | 1 |

The resulting PCR product was bound to the column matrix of the PCR purification kit (Qiagen, Hilden) and eluted with 300 mM NaP buffer, pH 7.0, 0.5 mM EDTA, 0.04% SDS. The DNA was denatured for 5 minutes in a boiling water bath and subsequently renatured for 24 hours at 60oC. 50 μl of the DNA were applied to a hydroxylapatite column and the column was washed 3 times with 1 ml of 10 mM NaP buffer, pH 6.8. The bound single-stranded DNA was eluted with 130 mM NaP buffer, pH 6.8, precipitated with ethanol and dissolved in 40 μl of water. 20 μl of growth were used for a further PCR amplification as described above. After further ssDNA concentration, a third PCR amplification was carried out as described above.

The plant transformation vector for taking up the cDNA population which had been generated as described above was generated via restriction enzyme cleavage of the vector pUC18 with SbfI and BamHI, purification of the vector fragment followed by filling up the overhangs with Pfu DNA polymerase and relegation with T4 DNA ligase (Stratagene). The resulting construct is hereinbelow termed pUC18SbfI-.

The vector pBinAR was first cleaved with NotI, the ends were filled up and the vector was relegated, cleaved with SbfI, the ends were filled up and the vector was relegated and subsequently cleaved with EcoRI and HindIII. The resulting fragment was ligated into a derivative of the binary plant transformation vector pPZP (Hajdukiewicz, P, Svab, Z, Maliga, P., (1994) Plant Mol Biol 25:989-994) which makes possible the transformation of plants by means of agrobacterium and mediates kanamycin resistance in transgenic plants. The construct generated thus is hereinbelow termed pSun12/35S.

pUC18Sbfl- was used as template in a polymerase chain reaction (PCR) with the oligonucleotides V1 and V2 (see Table 2) and Pfu DNA polymerase. The resulting fragment was ligated into the SmaI-cut psun12/35S, giving rise to pSunblues2. Following cleavage with NotI, dephosphorylation with shrimp alkaline phosphatase (Roche Diagnostics, Mannheim) and purification of the vector fragment, pSunblues2 was ligated with the normalized, likewise NotI-cut cDNA population. Following transformation into E. coli XI-1 blue (Stratagene), the resulting clones were deposited into microtiter plates. The binary cDNA library contains cDNAs in “sense”—and in “antisense” orientation under the control of the cauliflower mosaic virus 35S promoter, and, after transformation into tobacco plants, these cDNAs can, accordingly, lead to “cosuppression” and “antisense” effects.

TABLE 2
Oligonucleotides used

| Oligo- | |
|---|---|
| nucleotide | Nucleic acid sequence |
| N1 | 5′-AGAATTCGCGGCCGCT-3′ |
| | (SEQ ID NO:23) |
| V1 | 5′-CTCATGCGGCCGCGCGCAACGCAATTAATGTG-3′ |
| (PWL93not) | (SEQ ID NO:24) |
| V2 | 5′-TCATGCGGCCGCGAGATCCAGTTCGATGTAAC-3′ |
| (pWL92) | (SEQ ID NO:25) |
| G1 (35S) | 5′-GTGGATTGATGTGATATCTCC-3′ |
| | (SEQ ID NO:26) |
| G2 (OCS) | 5′-GTAAGGATCTGAGCTACACAT-3′ |
| | (SEQ ID NO:27) |

### EXAMPLE 2

### Transformation and Analysis of Tobacco Plants

Selected clones of the binary cDNA library were transformed into Agrobacterium tumefaciens C58C1:pGV2260 and (Deblaere et al., Nucl. Acids. Res. 13(1984), 4777-4788) and incubated with Streptomycin/Spectinomycin selection. The material used for the transformation of tobacco plants (Nicotiana tabacum cv. Samsun N N) with one of the binary clones as depicted in table 3 was an overnight culture of a positively transformed agrobacterial colony diluted with YEB medium to OD600=0.8-1.6. Leaf discs of sterile plants (approx. 1 cm2 each) were incubated for 5-10 minutes with a 1:50 agrobacterial dilution in a Petri dish. This was followed by incubation in the dark for 2 days at 25° C. on Murashige-Skoog medium (Physiol. Plant. 15(1962), 473) supplemented with 2% sucrose (2MS medium) and 0.8% Bacto agar. The cultivation was continued after 2 days at a 16-hour-lightV8-hour-darkness photoperiod and continued in a weekly rhythm on MS medium supplemented with 500 mg/l Claforan (cefotaxime sodium), 50 mg/l kanamycin, 1 mg/l benzylaminopurin (BAP), 0.2 mg/l naphthylacetic acid and 1.6 g/l glucose. Regenerated shoots were transferred onto an MS medium supplemented with kanamycin and Claforan. Transgenic plants of lines as depicted in table 3 were generated in this manner.

TABLE 3
Plant lines generated

| Partial cDNA | | | |
|---|---|---|---|
| with phenotype | | Corresponding | |
| in transgenic | | full length | |
| tobacco | Plant line | cDNA | Function |
| SEQ ID NO: 20 | E_0000013511 | SEQ ID NO: 3 | ClpP2 protease |
| SEQ ID NO: 19 | E_0000008893 | SEQ ID NO: 11 | ClpP5 = ClpR1 |
| | | | protease |
| SEQ ID NO: 21 | E_0000012393 | — | ClpP6 protease |
| — | — | SEQ ID NO: 17 | ClpP-like |
| | | | protease |

The integration of the clone cDNA into the genome of the transgenic lines was detected via PCR with the oligonucleotides G1 and G2 (see Table 2) and genomic DNA prepared from the transgenic lines in question. To this end, TAKARA Taq DNA polymerase was preferably employed for this purpose, following the manufacturer's instructions (MoBiTec, Gottingen). The cDNA clone of the binary cDNA library, which clone had been used for the transformation, acted as template for a PCR reaction as the positive control. PCR products with an identical size or, if appropriate, identical cleavage patterns which were obtained after cleavage with a variety of restriction enzymes acted as proof that the corresponding cDNA had been integrated. In this manner, the insert of clones were detected in the respective transgenic plant lines (as depicted in table 3) with the belowmentioned phenotypes.

After the shoots had been transferred into soil, the plants were observed for 2-20 weeks in the greenhouse for the manifestation of phenotypes. It emerged that transgenic plants of lines E_{—}0000012393, E_{—}0000013511 and E_{—}0000008893 were similar in phenotype. The plants showed severe chlorosis and concomitant growth retardation with respect to wild type plants after 2 weeks.

### EXAMPLE 3

### Sequence Analysis of the Clones

SEQ ID NO:19 was fully sequenced and used for the detection of the corresponding full length clone SEQ ID NO:11. SEQ ID NO:11 is identical to nt002050071r SEQ ID NO:19 in the overlapping region. An open reading frame of 867 nt (pos. 2-1162) encodes for 387 amino acids (SEQ ID NO: 4) with highest identity to-ClpR1 from Arabidopsis thaliana-. Sequence homology suggests that the 5′-ends of SEQ ID NO:11 and ClpR1 from Arabidopsis thaliana are very diverse and that nt006066004r is close to being full size with respect to ClpR1. MS-Analysis of isolated ClpP Proteins from Arabidopsis indicate, that the mature ClpR1 is several kDal shorter as suggested by the cDNA Sequence (Peltier et al. 2001, The Journal of Biological Chemistry 276, 99.16318-16327).

SEQ ID NO:20 was fully sequenced and used for the detection of the corresponding full length clone, SEQ ID NO: 3. SEQ ID NO:3 is identical to SEQ ID NO:20 in the overlapping region. An open reading frame of 867 nt (pos. 11-877) encodes for 289 amino acids (SEQ ID NO:4) with highest identity to ClpP2 from Arabidopsis thaliana.

SEQ ID NO:21 was fully sequenced. The partial cDNA Sequence of 602 nt contains an open reading frame of 186 nt (nt 8-193) encoding for 62 amico acids (SEQ ID NO:22). This partial polypeptide shows highest identity to ClpP6 from Arabidopsis thaliana (SEQ ID NO:9)

A further ClpP-homolog cDNA of 906 nt (SEQ ID NO:17) was identified. An open reading frame of 711 nt (pos. 45-755) encodes for 237 amino acids (SEQ ID NO: 18) with highest identity to a ClpP-like protein from Arabidopsis thaliana (GeneBank Acc. No. AK118523).

Thus, it was shown for the first time and in a surprising manner that the natural expression of nuclear encoded Clp protease encoding genes is essential for plants and that reduced expression leads to damage as depicted by the phenotypes mentioned in Example 2 demonstrating the suitability of nuclear encoded Clp-proteases as target for herbicides.

### EXAMPLE 4

### Expression in E. coli

In order to generate active protein with nuclear encoded Clp-protease activity fragments of SEQ ID NO:11, -SEQ ID NO:3 and of SEQ ID NO:9 were subcloned into the expression vector pQE60 (Quiagen, Hilden, Germany). To this end the oligonucleotides displayed in tab. 4 where used to amplify via polymerase chain reaction cDNA fragments that contain NcoI and BgIll restriction sites. The PCR was carried out in 36 cycles following standard conditions (for example as described by Sambrook, J. et al. (1989) “Molecular cloning: A laboratory manual”, Cold Spring Harbor Laboratory Press), the annealing temperatures being between 45 and 55° C. and the polymerization time being in each case 60 seconds per 1000 bp. Cutting the cDNA fragments with NcoI and BglII restriction enzymes and ligation into pQE60 cut with the same enzymes delivered expression plasmids that were transformed into E. coli. Expression was performed in E. coli TOP 10F strains (Invitrogen, Karlsruhe, Germany) following induction with IPTG. Standard protocols (Invitrogen) were followed.

Enzyme preperations were achieved by breaking cells in a French-Press in 100 mM Tris/HCl, pH 7.4, 2.5 mM EDTA, 1% Triton X-100.

The expression products were purified by affinity chromatography on Ni-agarose where appropriate. The manufacturer's instructions were followed (Qiagen).

TABLE 4

| | |
|---|---|
| Construct | Primer (Nucleic acid sequence) |

| | |
|---|---|
| NT_ClpR1¹⁾ | 5′-TATACCATGGATTTGCCATCTTTG-3′ |
| | (SEQ ID NO:28) |
| | 5′-ATAGATCTCACCTGGAGCCAG-3′ |
| | (SEQ ID NO:29) |
| Nt_ClpP2¹⁾ | 5′-GAGCCCATGGCAAGAGGAG-3′ |
| | (SEQ ID NO:30) |
| | 5′-ATAGATCTTTCTAGCTTGAACC-3′ |
| | (SEQ ID NO:31) |
| AT_ClpP6²⁾ | 5′-TCAGCCATGGCCCCTGGAGGAC-3′ |
| | (SEQ ID NO:32) |
| | 5′-TAAGATCTTCAGTATTCTGTTTCC-3′ |
| | (SEQ ID NO:33) |

1) Template: Nicotiana tabacum cDNA library
2) Template: Arabidopsis thaliana cDNA library

### EXAMPLE 5

### Activity Assay

Isolated ClpP activity can be measured as described (Woo et al. 1989 The Journal of Biological Chemistry 264, pp. 2088-2091) by using fluoregenic substrates such as N-Suc-Leu-Tyr-(7-amino-4-methylcoumarine) (SLT-AMC). The proteolytic cleavage deliberates 7-amino-4-methylcoumarin, which can be detected fluorimetrically (emission at 460 nm by exitation at 390 nm).

Standard assays contain: 50 mM Tris/HCl, pH 8.0, 25 mM MgCl₂, 1 mM SLT-AMC and 1-100 μg ClpP Enzyme.

The assay is suitable in for high throughput screening in 96well and 384 well format.

Screening According to the Above Mentioned Assay Provided the Following Compounds of the Formula:

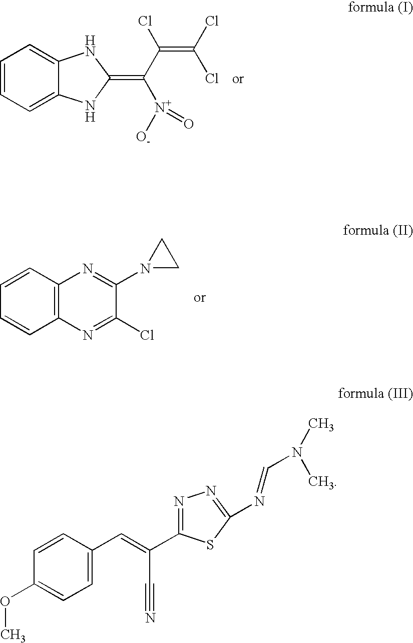

| compound of formula | IC50 |
|---|---|
| (I) | 2.3E−05 |
| (II) | 1.9E−05 |
| (III) | 2.5E−05 |

Sequence Listing

| Sequence | Function | Organism |
|---|---|---|
| SEQ ID NO: 1 (nucleic acid sequence) | ClpP1 | Arabidopsis thanliana |
| SEQ ID NO: 2 (amino acid sequence) | ClpP1 | Arabidopsis thanliana |
| SEQ ID NO: 3 (nucleic acid sequence) | ClpP2 | Nicotiana tabacuum |
| SEQ ID NO: 4 (amino acid sequence) | ClpP2 | Nicotiana tabacuum |
| SEQ ID NO: 5 (nucleic acid sequence) | ClpP3 | Arabidopsis thanliana |
| SEQ ID NO: 6 (amino acid sequence) | ClpP3 | Arabidopsis thanliana |
| SEQ ID NO: 7 (nucleic acid sequence) | ClpP4 | Arabidopsis thanliana |
| SEQ ID NO: 8 (amino acid sequence) | ClpP4 | Arabidopsis thanliana |
| SEQ ID NO: 9 (nucleic acid sequence) | ClpP6 | Arabidopsis thanliana |
| SEQ ID NO: 10 (amino acid sequence) | ClpP6 | Arabidopsis thanliana |
| SEQ ID NO: 11 (nucleic acid sequence) | ClpR1 | Nicotiana tabacuum |
| SEQ ID NO: 12 (amino acid sequence) | ClpR1 | Nicotiana tabacuum |
| SEQ ID NO: 13 (nucleic acid sequence) | ClpR3 | Arabidopsis thanliana |
| SEQ ID NO: 14 (amino acid sequence) | ClpR3 | Arabidopsis thanliana |
| SEQ ID NO: 15 (nucleic acid sequence) | ClpR4 | Arabidopsis thanliana |
| SEQ ID NO: 16 (amino acid sequence) | ClpR4 | Arabidopsis thanliana |
| SEQ ID NO: 17 (nucleic acid sequence) | ClpP like | Arabidopsis thanliana |
| SEQ ID NO: 18 (amino acid sequence) | ClpP like | Arabidopsis thanliana |
| SEQ ID NO: 19 (nucleic acid sequence) | ClpR1 | Nicotiana tabacuum |
| (fragment) | | |
| SEQ ID NO: 20 (nucleic acid sequence) | ClpP2 | Nicotiana tabacuum |
| (fragment) | | |
| SEQ ID NO: 21 (nucleic acid sequence) | ClpP6 | Nicotiana tabacuum |
| (fragment) | | |
| SEQ ID NO: 22 (amino acid sequence) | ClpP6 | Nicotiana tabacuum |
| (fragment) | | |
| SEQ ID NO: 23-33: Primer (nucleic acid sequences) | | |

1. A method for identifying herbicides comprising utilizing a nuclear encoded Clp-protease.

2. The method as claimed in claim 1, wherein the Clp-protease is
a) selected from the group consisting of ClpP1-protease, ClpP2-protease, ClpP3-protease, ClpP4-protease and ClpP6-protease; or
b) selected from the group consisting of ClpR1-protease, ClpR3-protease, and ClpR4-protease; or
c) ClpP-like-protease.

3. An isolated plant nucleic acid sequence encoding a ClpP2-protease comprising:
a) a nucleic acid sequence with the nucleic acid sequence shown in SEQ ID NO:3, or
b) a nucleic acid sequence which, owing to the degeneracy of the genetic code, can be deduced from the amino acid sequence shown in SEQ ID NO:4 by backtranslating, or
c) a functional equivalent of nucleic acid sequence shown in SEQ ID NO:3 which has an identity with SEQ ID NO:3 of has at least 66%.

4. An isolated plant nucleic acid sequence encoding a ClpR1-protease comprising:
a) a nucleic acid sequence with the nucleic acid sequence shown in SEQ ID NO: 11, or
b) a nucleic acid sequence which, owing to the degeneracy of the genetic code, can be deduced from the amino acid sequence shown in SEQ ID NO:12 by backtranslating, or
c) a functional equivalent of nucleic acid sequence shown in SEQ ID NO:11 which has an identity with SEQ ID NO:11 of at least 69%.

5. A plant nucleic acid sequence encoding a ClpP-like-protease comprising:
a) a nucleic acid sequence with the nucleic acid sequence shown in SEQ ID NO: 17, or
b) a nucleic acid sequence which, owing to the degeneracy of the genetic code, can be deduced from the amino acid sequence shown in SEQ ID NO: 18 by backtranslating, or
c) a functional equivalent of nucleic acid sequence shown in SEQ ID NO:17 which has an identity with SEQ ID NO:17 of at least 67%.

6. A polypeptide with the activity of a nuclear encoded Clp-protease, encoded by a nucleic acid molecule as claimed in claim 3.

7. An expression cassette comprising
genetic control sequences in operable linkage with a nucleic acid sequence as claimed in claim 3,
and optionally one or more additional functional elements.

8. A vector comprising an expression cassette as claimed in claim 7.

9. A transgenic organism comprising at least one nucleic acid sequence as claimed in claim 4, selected from the group consisting of bacteria, yeasts, fungi, animals, and plants.

10. A method for identifying substances with herbicidal activity, comprising the following steps:
i. bringing a nuclear encoded Clp-protease into contact with one or more test compounds under conditions which permit the test compound(s) to bind to a nucleic acid molecule encoding a Clp-protease or to the nuclear encoded Clp-protease, and
ii. detecting whether the test compound binds to the Clp-protease, or
iii. detecting whether the test compound reduces or blocks the enzymatic or biological activity of the Clp-protease, or
iv. detecting whether the test compound reduces or blocks the transcription, translation or expression of the Clp-protease.

11. The method as claimed in claim 10, wherein the Clp-protease is
a) selected from the group consisting of ClpP1-protease, ClpP2-protease, ClpP3-protease, ClpP4-protease and ClpP6-protease; or
b) selected from the group consisting of ClpR1-protease, ClpR3-protease, and ClpR4-protease; or
c) ClpP-like-protease.

12. The method as claimed in claim 10, wherein
a) the ClpP1-protease is encoded by a nucleic acid sequence which comprises:
i) a nucleic acid sequence with the nucleic acid sequence shown in SEQ ID NO:1, or
ii) a nucleic acid sequence which, owing to the degeneracy of the genetic code, can be deduced from the amino acid sequence shown in SEQ ID NO:2 by back translating, or
iii) a functional equivalent of nucleic acid sequence shown in SEQ ID NO:1 which has an identity with SEQ ID NO:1 of at least 50%;
b) the ClpP2-protease is encoded by a nucleic acid sequence which comprises:
i) a nucleic acid sequence with the nucleic acid sequence shown in SEQ ID NO:3, or
ii) a nucleic acid sequence which, owing to the degeneracy of the genetic code, can be deduced from the amino acid sequence shown in SEQ ID NO:4 by back translating, or
iii) a functional equivalent of nucleic acid sequence shown in SEQ ID NO:3 which has an identity with SEQ ID NO:3 of at least 50%;
c) the ClpP3-protease is encoded by a nucleic acid sequence which comprises:
i) a nucleic acid sequence with the nucleic acid sequence shown in SEQ ID NO:5, or
ii) a nucleic acid sequence which, owing to the degeneracy of the genetic code, can be deduced from the amino acid sequence shown in SEQ ID NO:6 by back translating, or
iii) a functional equivalent of nucleic acid sequence shown in SEQ ID NO:5 which has an identity with SEQ ID NO:5 of at least 50%;
d) the ClpP4-protease is encoded by a nucleic acid sequence which comprises:
i) a nucleic acid sequence with the nucleic acid sequence shown in SEQ ID NO:7, or
ii) a nucleic acid sequence which, owing to the degeneracy of the genetic code, can be deduced from the amino acid sequence shown in SEQ ID NO:8 by back translating, or
iii) a functional equivalent of nucleic acid sequence shown in SEQ ID NO:7 which has an identity with SEQ ID NO:7 of at least 50%;
e) the ClpP6-protease is encoded by a nucleic acid sequence which comprises:
i) a nucleic acid sequence with the nucleic acid sequence shown in SEQ ID NO:9, or
ii) a nucleic acid sequence which, owing to the degeneracy of the genetic code, can be deduced from the amino acid sequence shown in SEQ ID NO:10 by back translating, or
iii) a functional equivalent of nucleic acid sequence shown in SEQ ID NO:9 which has an identity with SEQ ID NO:9 of at least 50%;
f) the ClpR1-protease is encoded by a nucleic acid sequence which comprises:
i) a nucleic acid sequence with the nucleic acid sequence shown in SEQ ID NO: 11, or
ii) a nucleic acid sequence which, owing to the degeneracy of the genetic code, can be deduced from the amino acid sequence shown in SEQ ID NO:12 by back translating, or
iii) a functional equivalent of nucleic acid sequence shown in SEQ ID NO:11 which has an identity with SEQ ID NO:11 of at least 50%;
g) the ClpR3-protease is encoded by a nucleic acid sequence which comprises:
i) a nucleic acid sequence with the nucleic acid sequence shown in SEQ ID NO:13, or
ii) a nucleic acid sequence which, owing to the degeneracy of the genetic code, can be deduced from the amino acid sequence shown in SEQ ID NO:14 by back translating, or
iii) a functional equivalent of nucleic acid sequence shown in SEQ ID NO:13 which has an identity with SEQ ID NO:13 of at least 50%;
h) the ClpR4-protease is encoded by a nucleic acid sequence which comprises:
i) a nucleic acid sequence with the nucleic acid sequence shown in SEQ ID NO:15, or
ii) a nucleic acid sequence which, owing to the degeneracy of the genetic code, can be deduced from the amino acid sequence shown in SEQ ID NO:16 by back translating, or
iii) a functional equivalent of nucleic acid sequence shown in SEQ ID NO:15 which has an identity with SEQ ID NO:15 of at least 50%;
i) the ClpP like-protease is encoded by a nucleic acid sequence which comprises:
i) a nucleic acid sequence with the nucleic acid sequence shown in SEQ ID NO:17, or
ii) a nucleic acid sequence which, owing to the degeneracy of the genetic code, can be deduced from the amino acid sequence shown in SEQ ID NO:18 by back translating, or
iii) a functional equivalent of nucleic acid sequence shown in SEQ ID NO:17 which has an identity with SEQ ID NO:17 of at least 50%;

13. A The method as claimed in claim 10, wherein a test compound is selected which reduces or blocks the enzymatic or biological activity of Clp-protease.

14. The method as claimed in claim 10, wherein
i. either Clp-protease is expressed in a transgenic organism or an organism which naturally contains Clp-protease is grown,
ii. the Clp-protease of step i) is brought into contact with a test compound in the cell digest of the transgenic or nontransgenic organism, in partially purified form or in homogeneously purified form, and
iii. selecting a test compound which reduces or blocks the enzymatic activity of the Clp-protease of step a).

15. The method as claimed in claim 10, which comprises the following steps:
i. generating a transgenic organism comprising a nucleic acid sequence encoding Clp-protease, wherein Clp-protease is expressed recombinantly;
ii. applying a test substance to the transgenic organism of i) and to a nontransgenic organism of the same genotype,
iii. determining the growth or the viability of the transgenic plant and the nontransgenic plant after application of the test substance, and
iv. selecting a test substance which bring about a reduced growth of the nontransgenic plant in comparison with the growth of the transgenic plant.

16. The method as claimed in claim 15, which is carried out in a plant organism, a cyanobacterium or proteobacterium.

17. A method for identifying substances with growth-regulatory activity, which comprises the following steps:
i. generating a transgenic plant comprising a nucleic acid sequence Clp-protease, wherein Clp-protease is expressed recombinantly;
ii. applying a test substance to the transgenic plant of i) and to a nontransgenic plant of the same variety,
iii. determining the growth or the viability of the transgenic plant and the nontransgenic plant after application of the test substance, and
iv. selecting a test substance which bring about a reduced growth of the nontransgenic plant in comparison with the growth of the transgenic plant.

18. The method as claimed in claim 10, wherein the substances are identified by a high-throughput screening method.

19. A support comprising one or more of the nucleic acid molecules as claimed in claim 3.

20. The method as claimed in claim 10, wherein the substances are identified by High-Throughput Screening using a support comprising one or more nucleic acid molecules comprising a plant nucleic acid sequence encoding a ClpP2-protease comprising:
a) a nucleic acid sequence with the nucleic acid sequence shown in SEQ ID NO:3, or
b) a nucleic acid sequence which, owing to the degeneracy of the genetic code, can be deduced from the amino acid sequence shown in SEQ ID NO:4 by backtranslating, or
c) a functional equivalent of nucleic acid sequence shown in SEQ ID NO:3 which has an identity with SEQ ID NO:3 of at least 66%.

21. A method for controlling undesired vegetation and/or for regulating the growth of plants which comprises utilizing a compound with herbicidal activity, identified by the method as claimed in claim 10.

22. A method for controlling undesired vegetation and/or for regulating the growth of plants which comprises utilizing a compound with growth-regulatory activity, identified by the method as claimed in claim 17.

23. A method for the preparation of an agrochemical composition, which comprises
a) identifying a compound with herbicidal activity by the method as claimed in claim 10, and
b) formulating this compound together with suitable auxiliaries to give crop protection products with herbicidal or growth-regulatory activity.

24. A method for controlling undesired vegetation and/or for regulating the growth of plants comprising utilizing at least one Clp-protease inhibitor identified by the method as claimed in claim 10.

25. A method for controlling undesired vegetation and/or for regulating the growth of plants comprising treating said undesired vegetation or plants with a herbicide, wherein said herbicide is a compound which is a inhibitor of a Clp-protease.

26. A Clp-protease inhibitor of the formula:

27. A polypeptide with the activity of a nuclear encoded Clp-protease, encoded by a nucleic acid molecule as claimed in claim 4.

29. A transgenic organism comprising an expression cassette as claimed in claim 7, selected from the group consisting of bacteria, yeasts, fungi, animals, and plants.

30. A transgenic organism comprising a vector as claimed in claim 8, selected from the group consisting of bacteria, yeasts, fungi, animals, and plants.

31. The method as claimed in claim 17, wherein the substances are identified by high-throughput screening.

32. A support comprising one or more expression cassettes as claimed in claim 7.

33. A method for the preparation of an agrochemical composition, which comprises
a) identifying a compound with growth-regulatory activity as claimed in claim 17, and
b) formulating this compound together with suitable auxiliaries to give crop protection products with herbicidal or growth-regulatory activity.

34. An expression cassette comprising genetic control sequences in operable linkage with a nucleic acid sequence as claimed in claim 4 and optionally one or more additional functional elements.

35. An expression cassette comprising genetic control sequences in operable linkage with a nucleic acid sequence as claimed in claim 5 and optionally one or more additional functional elements.

36. The expression cassette of claim 7, wherein the additional functional elements are selected from the group consisting of reporter genes, replication origins, selection markers, affinity tags, and sequences which target products into apoplasts, plastids, vacuoles, mitochondria, peroxisomes, endoplasmatic reticulum (ER), or cytosol.
